(19) United States
(12) Patent Application Publication
(10) Pub. No.: US 2022/0275432 A1
(43) Pub. Date: Sep. 1, 2022

(54) PROBE-INDUCED HETERODUPLEX MOBILITY ASSAY

(71) Applicants: UNIVERSITÄT ZÜRICH, Zürich (CH); PUBLIC UNIVERSITY CORPORATION YOKOHAMA CITY UNIVERSITY, Yokohama-shi, Kanagawa (JP)

(72) Inventors: Hiroyuki KAKUI, Yokohama (JP); Kentaro K. SHIMIZU, Zurich (CH); Misako YAMAZAKI, Zurich (CH)

(73) Assignees: UNIVERSITÄT ZÜRICH, Zürich (CH); PUBLIC UNIVERSITY CORPORATION YOKOHAMA CITY UNIVERSITY, Yokohama-shi, Kanagawa (JP)

(21) Appl. No.: 17/632,519

(22) PCT Filed: Aug. 10, 2020
(86) PCT No.: PCT/EP2020/072434

§ 371 Date: Feb. 3, 2022

(30) Foreign Application Priority Data
Aug. 8, 2019 (EP) 19190891.2

(51) Int. Cl. C12Q 1/6827; G01N 27/447

(52) U.S. Cl. CPC C12Q 1/6827; G01N 27/44704; C12Q 2600/156

(57) ABSTRACT

The present invention relates to a method for distinguishing a first nucleic acid sequence from a second nucleic acid sequence by electrophoresis. The first nucleic acid comprises a first common sequence tract, a variable sequence tract and a second common sequence tract and the second nucleic acid comprises a first common sequence tract, optionally an variable sequence tract and a second common sequence tract. The first and the second nucleic acid sequence is contacted with a probe sequence that is reverse complementary to the first and second common sequence tract under conditions allowing the hybridization of the probe sequence to the first and second nucleic acid sequence, thereby forming a first probe hybrid and a second probe hybrid. Subsequently, the first and second probe hybrids are submitted to electrophoresis to detect the electrophoretic mobility of the first and second probe hybrid.

### BACKGROUND

There are increasing demands to detect 1 bp differences in molecular biology, because of the recent advancement of gene-editing technology (i.e. ZFN/TALEN/CRIPSR) based on double strand break (DSB). These DSB can stimulate non-homologous end joining (NHEJ) at the targeted genome sequence and produce 1 bp insertion or deletion (indel) mutation. Researchers are often interested in these 1 bp indel mutants resulting in a frame shift null mutation. A large number of genotyping experiments would be necessary first to identify such mutations from a screening population, and once the mutation is identified, large-scale genotyping homozygotes and heterozygote may be necessary for subsequent analysis. Such experiments are common in many organisms (Human; Mali et al., 2013 Science/Mouse; Wang et al Cell 2013/monkey; Wan et al., 2015 Cell Res 2014/C. elegans; Friedland Nat Methods 2013/Dorosophila; Venken et al., Dev Biol., 2016 Zebrafish; Hwang et al., 2013 Nat biotech./Athal Nbenthamiana; Li et al., Nat biotech 2013/sorghum rice; Jiang et al., NAR 2013/wheat; Upadhyay et al., G3 2013). Methods for detecting a few base pair differences are developed by many researches, for example, sanger or deep sequencing, restriction fragment length polymorphism (RFLP) analysis (Urnov et al., 2005 nature), DNA melting analysis (Dahlem et al., 2012 PLoS Genet), T7 endonuclease I assay (Kim et al., 2009 Genome Res), Cel-1 assay (Ueta et al., 2017 Scientific Rep), fluorescent polymerase chain reaction (PCR) (Kim et al., 2011 Nat methods) and analysis based on RNA-guided endonucleases and restriction fragment length polymorphism (RGEN-RFLP) (Kim et al., 2014 Nat Comn). However, each technique has advantages and disadvantages. For example, Sanger or deep sequencing can identify DNA sequence at 1 bp resolution but they require cost and time. RFLP analysis could achieve 1 bp resolution when the researchers already knew the information of sequences to be distinguished and can design the assay with an existing restriction enzyme. With this condition, RFLP is not suitable for mutant screening. DNA melting analysis, T7 endonuclease I assay, Cel-1 assay, fluorescent PCR and RGEN-RFLP are not always successful to obtain 1 bp resolution and/or need special chemicals/proteins/devices.

Heteroduplex mobility assay (HMA) is also a method to detect the small base pair difference (Kumeda and Asao 2001, Appl Environ Microbiol, Ota et al., 2013 Genes Cells, Ansai et al., 2014 Dev Growth Differ, Bhattacharyya and Lilley, 1989 NAR). HMA is consisted of 3 simple steps; 1) PCR, 2) denaturation/re-annealing and 3) electrophoresis (FIG. 1). However, the resolution of HMA is typically 3 or more base pairs (Ota et al., 2013 Genes Cells, Ansai et al., 2014 Dev Growth Differ, Bhattacharyya and Lilley, 1989 NAR), and thus it is normally difficult to distinguish 1 bp difference using HMA (Sugano et al., 2017).

The present invention provides a novel method of detecting 1 bp different sequences by using synthesized oligo DNA sequence with artificially introduced insertion or deletion and PCR amplified double stranded DNA or short single strand DNA as probe. The inventors refer to this method as Probe-Induced HMA (PRIMA) herein. PRIMA has a broad range of application in genome editing of diverse species.

### SUMMARY OF THE INVENTION

A first aspect of the invention relates to a method for distinguishing a first nucleic acid sequence from a second nucleic acid sequence by electrophoresis, wherein the first nucleic acid sequence S1 comprises
a first 5′ common sequence tract C1, and
a first, optional, variable sequence tract V1 of 1, 2, 3, 4, 5, 6, 7, 8, 9 or 10 nucleotides, immediately adjacent in 3′ direction to C1; and
a first 3′ common sequence tract C2 positioned in 3′ direction of C1;
   the second nucleic acid sequence S2 comprises
a second 5′ common sequence tract C1′, and
a second, optional, variable sequence tract V2 of 1, 2, 3, 4, 5, 6, 7, 8, 9 or 10 nucleotides, immediately adjacent in 3′ direction to C1′; and
a second 3′ common sequence tract C2′ positioned in 3′ direction of C1′;
   and wherein
the first 5′ common sequence tract C1 is identical to the second 5′ common sequence tract C1′, or
C1′ is 1 to 9 nucleotides shorter at the 3′ end than C1 and C1′ is identical to C1 from the 5′ end of C1/C1′; and
the first 3′ common sequence tract C2 is identical to the second 3′ common sequence tract C2′, or
C2′ is 1 to 9 nucleotides shorter at the 5′ end than the first 3′ common sequence tract C2 and C2′ is identical to C2 from the 3′ end of C2/C2′; and
   with the proviso that S1 and S2 with respect to their sequence tracts C1-V1-C2 and C1′-V2-C2′ differ from each other in length by 1, 2, 3, 4, 5, 6, 7, 8 or 9 nucleotides; said method comprising:
   contacting the first nucleic acid sequence and the second nucleic acid sequence with a probe sequence P, said probe sequence consisting, in 5′ to 3′ orientation, of a sequence RC2 that is reverse complementary to the 3′ common sequence tract C2 and a sequence RC1 that is reverse complementary to the 5′ common sequence tract C1,
   under conditions allowing the hybridization of the probe sequence to the first and second nucleic acid sequence, thereby forming a first probe hybrid and a second probe hybrid,
   and subsequently submitting the first and second probe hybrids to electrophoresis and detecting the electrophoretic mobility of the first and second probe hybrid.

The method aims to detect small variations between two nucleic acid sequences. For instance, the method may be applied after editing a nucleic acid sequence using the CRISPR/Cas system, which may induce non-homologous end joining at the targeted nucleic acid sequence, thereby producing an insertion or deletion of 1 base pair (bp) compared to the reference sequence.

In a typical approach, the sequence of the reference sequence and the edited sequence around the 1 bp mutation are amplified by standard PCR methods to provide said first nucleic acid sequence S1 (e.g. the sense strand of the PCR product of the reference sequence) and said nucleic acid sequence S2 (e.g. the sense strand of the PCR product of the edited sequence having a 1 bp mutation compared to the reference sequence) (FIG. 2).

Subsequently, the PCR products are denatured and incubated with a probe sequence P. The probe sequence anneals to the sequence S1 in two regions referred to as common sequence tracts, i.e. the probe sequence is antisense (reverse complementary) to the common sequence tracts of S1 and S2. The 5′ and 3′ common sequence tracts flank a variable region referred to as variable sequence tract, e.g. a sequence tract of 5 nucleotides (nt) around the mutation site. Upon hybridization of the nucleic acid sequence S1 and the probe sequence, the variable sequence tract of 5 nt will bulge out.

The same applies for the sequence S2. Also here, the probe sequence will hybridize to 5′ and 3′ common sequence tracts. Compared to the sequence S1, the variable sequence tract is one nucleotide longer (in case of a 1 bp insertion) or one nucleotide shorter (in case of a 1 bp deletion). Thus, 6 nt (insertion) or 4 nt (deletion) will bulge out.

When the S1-P-hybrid (first probe hybrid) and the S2-P-hybrid (second probe hybrid) are submitted to electrophoresis such as polyacrylamide gel electrophoresis or a high resolution electrophoresis machine (e.g. MultiNA or QIAxcel), the electrophoretic mobility of the first probe hybrid differs from the electrophoretic mobility of the second probe hybrid due to the different sizes of the bulges formed by the first variable sequence tract and the second variable sequence tract.

It is also possible that the probe sequence will bulge out. For example, a reference sequence S1 may comprise a first 5′ common sequence tract, a first 3′ common sequence tract and a variable sequence tract of e.g. 5 nt length. An edited nucleic acid sequence S2 may comprise a deletion of a few base pairs (e.g. 8 bp) compared to the reference sequence S1.

Thus, the 5′ common sequence tract C1′ of the edited sequence S2 is 3 nt shorter than the common sequence tract C1 of the reference sequence S1 (FIG. 3).

Upon hybridization to a probe sequence P, which consists of a sequence that is reverse complementary to C1 and C2, the variable sequence tract V1 will form a bulge of 5 nt. When the probe hybridizes with the edited sequence S2, the probe will form a bulge of 3 nt. Again, the electrophoretic mobility of the S1-P-hybrid differs from the electrophoretic mobility of the S2-P-hybrid when submitted to electrophoresis.

### DETAILED DESCRIPTION OF THE INVENTION

### Terms and Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The terms capable of forming a hybrid or hybridizing sequence in the context of the present specification relate to sequences that under the conditions typically existing within a gel employed for electrophoretic separation of polynucleotides, are able to bind selectively to their target sequence.

The term nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). Wherever reference is made herein to a hybridizing sequence, such hybridizing sequence may be composed of any of the above nucleotides, or mixtures thereof.

The term reverse complementary in the context of the present specification relates to a nucleotide sequence having a sequence, shown from 5′ to 3′, substantially complementary to, and capable of hybridizing to, a reference sequence. For example, if the reference sequence is 5′AATGC3′, the reverse complementary sequence thereto is 5′GCATT3′. “Complementary” is sometimes used synonymously to “reverse complementary”.

In the context of the present specification, the term hybridizing sequence encompasses a polynucleotide sequence comprising or essentially consisting of RNA (ribonucleotides), DNA (deoxyribonucleotides), phosphothioate deoxyribonucleotides, 2′-O-methyl-modified phosphothioate ribonucleotides, LNA and/or PNA nucleotide analogues.

### DETAILED DESCRIPTION

A first aspect of the invention relates to a method for distinguishing a first nucleic acid sequence from a second nucleic acid sequence by electrophoresis,

wherein
the electrophoretic mobility of the first nucleic acid sequence cannot be distinguished from
the electrophoretic mobility of the second nucleic acid sequence,
and wherein
the first nucleic acid sequence S1 comprises
a first 5′ common sequence tract C1, and
a first variable sequence tract V1 which can be of 1 to 10 nucleotides in length, immediately adjacent in 3′ direction to the first 5′ common sequence tract C1 and immediately adjacent in 5′ direction to the first 3′ common sequence tract C2; and
a first 3′ common sequence tract C2 positioned in 3′ direction of C1 and, if V1 is present, immediately adjacent in 3′ direction to the first variable sequence tract V1;
   the second nucleic acid sequence S2 comprises
a second 5′ common sequence tract C1′, and
a second, optional, variable sequence tract V2 which can be of 1 to 10 nucleotides in length, immediately adjacent in 3′ direction to the second 5′ common sequence tract C1′ and immediately adjacent in 5′ direction to the second 3′ common sequence tract C2′; and
a second 3′ common sequence tract C2′ positioned in 3′ direction of C1′ and, if V2 is present, immediately adjacent in 3′ direction to the second variable sequence tract V2;
   and wherein
the first 5′ common sequence tract C1 is identical to the second 5′ common sequence tract C1′, or
the second 5′ common sequence tract C1′ is 1 to 9 nucleotides shorter at the 3′ end than the first 5′ common sequence tract C1 and the second 5′ common sequence tract C1′ is identical to the first 5′ common sequence tract C1 from the 5′ end of C1/C1′ to the position −9 to −1 upstream (in 5′ direction) of the 3′ end; and
the first 3′ common sequence tract C2 is identical to the second 3′ common sequence tract C2′, or
the second 3′ common sequence tract C2′ is 1 to 9 nucleotides shorter at the 5′ end than the first 3′ common sequence tract C2 and the second 3′ common sequence tract C2′ is identical to the first 3′ common sequence tract C2 from the position +9 to +1 downstream (in 3′ direction) of the 5′ end to the 5′ end; and
if both the first 5′ common sequence tract and the first 3′ common sequence tract are identical to the second 5′ common sequence tract and the second 3′ common sequence tract, at least one of the first and second nucleic acid sequence comprises a first or second variable sequence tract; and
if the variable sequence tract is presence and C1 is identical to C1′ and C2 is identical to C2′, the first variable sequence tract is different in at least one position from the second variable sequence tract; and
in certain embodiments, the first variable sequence tract and/or the second variable sequence tract have a length of at least 2 nucleotides with the proviso that S1 and S2 with respect to their sequence tracts C1-V1-C2 and C1 ‘-V2-C2’ differ from each other in length by 10 nucleotides;
   said method comprising:
   contacting the first nucleic acid sequence and the second nucleic acid sequence with a probe sequence P, said probe sequence consisting, in 5′ to 3′ orientation, of a sequence RC2 that is reverse complementary to the 3′ common sequence tract C2 and a sequence RC1 that is reverse complementary to the 5′ common sequence tract C1, under conditions allowing the hybridization of the probe sequence to the first and second nucleic acid sequence, thereby forming a first probe hybrid and a second probe hybrid, and subsequently submitting the first and second probe hybrids to electrophoresis and detecting the electrophoretic mobility of the first and second probe hybrid.

According to one alternative of this aspect of the invention, the method for distinguishing a first nucleic acid sequence S1 from a second nucleic acid sequence S2 by electrophoresis employs sequences as follows
the first nucleic acid sequence S1 comprises
   a first 5′ common sequence tract C1, and
   a first variable sequence tract V1 of 1 to 10 nucleotides, immediately adjacent in 3′ direction to C1; and
   a first 3′ common sequence tract C2 positioned in 3′ direction of C1;
the second nucleic acid sequence S2 comprises
   a second 5′ common sequence tract C1′, and
   a second 3′ common sequence tract C2′ positioned in 3′ direction of C1′; and
   the first 5′ common sequence tract C1 is identical to the second 5′ common sequence tract C1′, and
   the first 3′ common sequence tract C2 is identical to the second 3′ common sequence tract C2′, and
S1 and S2 differ from each other in length, with respect to their sequence tracts C1-V1-C2 and C1-C2′, by ≤10 nucleotides.
The method comprises contacting the first nucleic acid sequence and the second nucleic acid sequence with a probe sequence P, said probe sequence consisting, in 5′ to 3′ orientation, of a sequence RC2 that is reverse complementary to the 3′ common sequence tract C2 and a sequence RC1 that is reverse complementary to the 5′ common sequence tract C1, under conditions allowing the hybridization of the probe sequence to the first and second nucleic acid sequence, thereby forming a first probe hybrid and a second probe hybrid, and subsequently submitting the first and second probe hybrids to electrophoresis and detecting the electrophoretic mobility of the first and second probe hybrid.

According to another alternative of this aspect of the invention, the method for distinguishing a first nucleic acid sequence S1 from a second nucleic acid sequence S2 by electrophoresis employs sequences as follows:
the first nucleic acid sequence S1 comprises
   a first 5′ common sequence tract C1, and
   a first variable sequence tract V1 of 1 to 10 nucleotides, immediately adjacent in 3′ direction to C1; and
   a first 3′ common sequence tract C2 positioned in 3′ direction of C1;
the second nucleic acid sequence S2 comprises
   a second 5′ common sequence tract C1′, and
   a second, variable sequence tract V2 of 1 to 10 nucleotides, immediately adjacent in 3′ direction to C1′; and
   a second 3′ common sequence tract C2′ positioned in 3′ direction of C1′;
and wherein
   the first 5′ common sequence tract C1 is identical to the second 5′ common sequence tract C1′, and
   the first 3′ common sequence tract C2 is identical to the second 3′ common sequence tract C2′, and
   S1 and S2 differ from each other in length, with respect to their sequence tracts C1-V1-02 and C1′-02′, by 10 nucleotides.
said method comprising:
As above, the method comprises contacting the first nucleic acid sequence and the second nucleic acid sequence with a probe sequence P, as defined above, under conditions allowing the hybridization of the probe sequence to the first and second nucleic acid sequence, and subsequently submitting the first and second probe hybrids to electrophoresis and detecting the electrophoretic mobility of the first and second probe hybrid.

In yet further alternatives of this aspect of the invention, the pairs of constant sequence tracts C1 and C1′ or C2 and C2′ may differ on their “far end”, i.e. the end that is opposite of the end where C1 is closest to C2 and C1′ closest to C2′:

In such alternative embodiments, C1′ is 1 to 9 nucleotides shorter at the 3′ end than C1 and C1′ is identical to C1 from the 5′ end of C1/C1′. Alternatively, C2′ is 1 to 9 nucleotides shorter at the 5′ end than the first 3′ common sequence tract C2 and C2′ is identical to C2 from the 3′ end of C2/C2′.

As described above, the sequences S1 and S2 may be obtained by performing standard PCR methods for example on a reference sequence and an edited sequence. Thus, the first nucleic acid sequence and the second nucleic acid sequence will have a length that is common to PCR products.

In certain embodiments, the length of the first nucleic acid sequence S1 and the length of the second nucleic acid sequence S2 is between 40 nucleotides and 3500 nucleotides.

In certain embodiments, the length of the first nucleic acid sequence S1 and the length of the second nucleic acid sequence S2 is between 60 nucleotides and 3500 nucleotides.

In certain embodiments, the length of the first nucleic acid sequence S1 and the length of the second nucleic acid sequence S2 is between 80 nucleotides and 3500 nucleotides.

In certain embodiments, the length of the first nucleic acid sequence S1 and the length of the second nucleic acid sequence S2 is between 100 nucleotides and 3500 nucleotides.

In certain embodiments, the length of the first nucleic acid sequence S1 and the length of the second nucleic acid sequence S2 is between 150 and 350 nucleotides, particularly between 150 nucleotides and 250 nucleotides.

In certain embodiments, the length of the first nucleic acid sequence S1 and the length of the second nucleic acid sequence S2 is between 180 nucleotides and 220 nucleotides.

The first and the second nucleic acid sequences S1 and S2 comprise common sequence tracts. When incubated with a probe sequence, the probe sequence will hybridize to the common sequence tracts.

According to the invention, the first and the second nucleic acid sequences S1 and S2 may start at their 5′ end with a common sequence tract and end at their 3′ end with a common sequence tract. Thus, except of a bulge region around the mutation site, the probe hybridizes over the entire length of S1 and S2. Such embodiment is also referred to as “pre-PRIMA”.

Alternatively, the first and the second nucleic acid sequences S1 and S2 may not start at their 5′ ends and at their 3′ ends with a common sequence tract. In this case, the probe does not hybridize to the sequence that is immediately adjacent in 5′ direction (upstream) to the 5′ common sequence tract and does not hybridize to the sequence that is immediately adjacent in 3′ direction (downstream) to the 3′ common sequence tract. Such embodiment is also referred to as “PRIMA”.

In certain embodiments, the first nucleic acid sequence S1 comprises at least 5 nucleotides immediately adjacent in 5′ direction to the first 5′ common sequence tract C1 and at least 5 nucleotides immediately adjacent in 3′ direction to the first 3′ common sequence tract C2 and the second nucleic acid sequence S2 comprises at least 5 nucleotides immediately adjacent in 5′ direction to second 5′ common sequence tract C1′ and at least 5 nucleotides immediately adjacent in 3′ direction to the second 3′ common sequence tract C2′.

In certain embodiments, the first nucleic acid sequence S1 comprises at least 35 nucleotides immediately adjacent in 5′ direction to the first 5′ common sequence tract C1 and at least 35 nucleotides immediately adjacent in 3′ direction to the first 3′ common sequence tract C2 and the second nucleic acid sequence S2 comprises at least 35 nucleotides immediately adjacent in 5′ direction to second 5′ common sequence tract C1′ and at least 35 nucleotides immediately adjacent in 3′ direction to the second 3′ common sequence tract C2′.

In certain embodiments, the first nucleic acid sequence S1 comprises at least 47 nucleotides, particularly 50 nucleotides, immediately adjacent in 5′ direction to the first 5′ common sequence tract C1 and at least 47 nucleotides, particularly 50 nucleotides, immediately adjacent in 3′ direction to the first 3′ common sequence tract C2 and the second nucleic acid sequence S2 comprises at least 47 nucleotides, particularly 50 nucleotides, immediately adjacent in 5′ direction to second 5′ common sequence tract C1′ and at least 47 nucleotides, particularly 50 nucleotides, immediately adjacent in 3′ direction to the second 3′ common sequence tract C2′.

The probe sequence may be obtained by PCR or oligonucleotide synthesis. When the method is performed on S1 and S2 sequences that do not start and end with a common sequence tract (“PRIMA”), the probe sequence is usually obtained by oligonucleotide synthesis. The probe is reverse complementary to the first 5′ common sequence tract C1 and the first 3′ common sequence tract C2.

In certain embodiments, the total length of the probe is between 18 and 80 nucleotides.

In certain embodiments, the total length of the sum of the first 5′ common sequence tract C1 and the first 3′ common sequence tract C2 is between 18 and 80 nucleotides.

In certain embodiments, the total length of the sum of the second 5′ common sequence tract C1 and the second 3′ common sequence tract C2 is between 18 and 80 nucleotides.

When the method is performed on S1 and S2 sequences that start and end with a common sequence tract (“pre-PRIMA”), the probe sequence is usually obtained by PCR. The probe is reverse complementary to the first 5′ common sequence tract C1 and the first 3′ common sequence tract C2.

In certain embodiments, the total length of the probe is between 18 and 3500 nucleotides, particularly between 40 and 80 nucleotides.

In certain embodiments, the total length of the sum of the first 5′ common sequence tract C1 and the first 3′ common sequence tract C2 is between 150 and 300 nucleotides.

In certain embodiments, the total length of the sum of the second 5′ common sequence tract C1 and the second 3′ common sequence tract C2 is between 200 and 250 nucleotides.

To ensure that a difference in electrophoretic mobility can be readily identified, the probe should be designed in such a way that a stable bulge region is formed. This means, that up- and downstream of the mutation site, the probe sequence should stably hybridize to the 5′ and 3′ common sequence tracts.

In certain embodiments, the ratio between the length of the first 5′ common sequence tract C1 and the length of the first 3′ common sequence tract C2 is between 1:7 to 7:1, wherein the minimum length of the first 5′ common sequence tract C1 and of the first 3′ common sequence tract C2 is 5, particularly 10, more particularly 20 nucleotides.

In certain embodiments, the ratio between the length of the first 5′ common sequence tract C1 and the length of the first 3′ common sequence tract C2 is between 3:5 and 5:3, wherein the minimum length of the first 5′ common sequence tract C1 and of the first 3′ common sequence tract C2 is 5, particularly 10, more particularly 20 nucleotides.

In certain embodiments, the ratio between the length of the first 5′ common sequence tract C1 and the length of the first 3′ common sequence tract C2 is 1:1, wherein the minimum length of the first 5′ common sequence tract C1 and of the first 3′ common sequence tract C2 is 5, particularly 10, more particularly 20 nucleotides.

In particular for the detection of a deletion or insertion of 1 bp in one of the sequences S1 or S2 with regard to the respective other sequence S2 or S1, bulge regions between 4 and 6 nucleotides are suitable. For example, a bulge having a length of 5 nucleotides (e.g. in the hybrid of a reference sequence and the probe) can be distinguished from a bulge having a length of 4 nucleotides (e.g. in the hybrid of an edited sequence with a 1 bp deletion and the probe) or from a bulge haven a length of 6 nucleotides (e.g. in the hybrid of an edited sequence with a 1 bp insertion and the probe). The bulge may be formed by the variable sequence tract of S1 and S2.

In certain embodiments, the first variable sequence tract V1 and the second variable sequence tract V2 have independently from each other a length between 4 and 10 nucleotides.

In certain embodiments, the first variable sequence tract V1 and the second variable sequence tract V2 have independently from each other a length between 4 and 6 nucleotides.

The sequences S1 and S2 can differ in length, for example S2 shows a deletion or insertion compared to S1. Alternatively or additionally, S1 and S2 may differ in the base sequence, e.g. ATGCTTC differs from ATGTCTC. Also a difference in composition might occur, e.g. S1 differs from S2 in a substitution such as ATCGTTC vs. ATCCTTC. To detect such differences, the probe may be designed in such a way that the mutation site is within a variable sequence tract flanked by common sequence tracts.

In certain embodiments, the first variable sequence tract V1 differs from the second variable sequence tract V2 in length (deletion/insertion) and/or the base sequence and/or composition of the first variable sequence tract V1 differs from the base sequence and/or composition of the second variable sequence tract V2 in at least one position (substitution).

In certain embodiments, the first variable sequence tract V1 differs from the second variable sequence tract V2 in length (deletion/insertion) and/or composition of the first variable sequence tract V1 differs from the composition of the second variable sequence tract V2 in at least one position (substitution).

In certain embodiments, the first variable sequence tract V1 differs from the second variable sequence tract V2 in length (deletion/insertion).

In certain embodiments, the length of the first variable sequence V1 tract differs from the length of the second variable sequence tract V2 in 10 nucleotides.

In certain embodiments, the length of the first variable sequence V1 tract differs from the length of the second variable sequence tract V2 in 2 nucleotides.

In certain embodiments, the length of the first variable sequence V1 tract differs from the length of the second variable sequence tract V2 in one nucleotide.

In certain embodiments, the composition of the first variable sequence tract V1 differs from the composition of the second variable sequence tract V2 in two positions, particularly in one position.

As described above, the method may be performed on sequences obtained by PCR. In this case, the first and second nucleic acid sequences S1 and S2 and/or the probe sequence are double stranded.

In certain embodiments, the first nucleic acid sequence S1 is hybridized to its reverse complementary sequence, and/or the second nucleic acid sequence S2 is hybridized to its reverse complementary sequence.

In certain embodiments, the probe sequence P is hybridized to its reverse complementary sequence.

In certain embodiments, the first probe hybrid and the second probe hybrid are obtained by applying a temperature above the melting point of the first and second nucleic acid sequence followed by applying a temperature below the melting point of the probe sequence.

An alternative aspect of the invention relates to a method for distinguishing a first nucleic acid sequence from a second nucleic acid sequence by electrophoresis,
wherein
the electrophoretic mobility of the first nucleic acid sequence cannot be distinguished from the electrophoretic mobility of the second nucleic acid sequence,
and wherein (see FIG. 16)
the first nucleic acid sequence comprises
   a first variable sequence tract,
   a first 5′ common sequence tract C1 immediately adjacent in 5′ direction to the first variable sequence tract, and
   a first 3′ common sequence tract C2 immediately adjacent in 3′ direction to the first variable sequence tract;
the second nucleic acid sequence comprises
   optionally a second variable sequence tract,
   a second 5′ common sequence tract C1′ that is identical to the first 5′ common sequence tract immediately adjacent in 5′ direction to the second variable sequence tract, and
   a second 3′ common sequence tract C2′ that is identical to the first 3′ common sequence tract immediately adjacent in 3′ direction to the second variable sequence tract;
and wherein
   the first variable sequence tract is different in at least one position from the second variable sequence tract; and
   the first variable sequence tract comprises a first sequence tract H and/or a first sequence tract A and optionally a first sequence tract U, wherein the first sequence tract H is identical to a second sequence tract H′ of the second variable sequence tract, the first sequence tract A is reverse complementary to a sequence tract RA of a probe sequence and the sequence tract U is unique to the first sequence, and
   the second variable sequence tract comprises the sequence tract H′ if the first variable sequence tract comprises the sequence tract H, and
   the second variable sequence tract may comprise a second sequence tract U′ that is unique to the second sequence,
said method comprising:
contacting the first nucleic acid sequence and the second nucleic acid sequence with a probe sequence, said probe sequence consisting, in 5′ to 3′ orientation, of a sequence RC2 that is reverse complementary to the 3′ common sequence tract C2 and a sequence RC1 that is reverse complementary to the 5′ common sequence tract C1, and optionally of a variable sequence tract RV that comprises a sequence tract RA that is reverse complementary to the sequence tract A and/or a sequence tract P that does not hybridize with any of the first variable sequence tract and the second variable sequence tract,
under conditions allowing the hybridization of the probe sequence to the first and second nucleic acid sequence, thereby forming a first probe hybrid and a second probe hybrid,
   and subsequently submitting the first and second probe hybrids to electrophoresis and detecting the electrophoretic mobility of the first and second probe hybrid.

### DESCRIPTION OF THE FIGURES

Sequences shown in the Figures are referenced separately immediately after the Figure description.

FIG. 1 shows an overview of HMA (A), prePRIMA (B) and PRIMA (C). HMA is difficult to produce detectable peak with heteroduplex mobility shift caused by 1 bp deference (a). On the other hand, prePRIMA (b) and PRIMA (c) are able to produce heteroduplex peaks from wild type and 1 bp indel sequences. WT; wild type, mt; mutant, Homo; Homozygous, Hetero; Heterozygous, sss; short single strand. Red lines of PCR fragment represent 1 bp insertion mutation. Green and red arrowheads indicate heteroduplex peak from wild type and mutant, respectively. Black circle above the electropherogram indicates mixture of homoduplex peak and undistinguishable heteroduplex peaks. Star indicates homoduplex peak.

FIG. 2 shows an exemplary sequence and probe design. Alignment of a first sequence (51), a second sequence (S2) and a probe (P). The first variable sequence tract V1 has a length of 5 nucleotides, the second variable sequence tract has a length of 4 nucleotides. X: no nucleotide (deletion with regard to V1); C1: first 5′ common sequence tract; C1′: second 5′ common sequence tract (identical to C1); C2: first 3′ common sequence tract; C2′ second 3′ common sequence tract (identical to C2); RC1: sequence reverse complementary to C1; RC2: sequence reverse complementary to C2; black lines: first and second sequence.

FIG. 3 shows an exemplary sequence and probe design. Alignment of a first sequence (S1), a second sequence (S2) and a probe (P). The first variable sequence tract V1 has a length of 5 nucleotides. X and Y: no nucleotide (deletion with regard to V1); C1: first 5′ common sequence tract; C1′: second 5′ common sequence tract (3 nucleotides shorter than C1); C2: first 3′ common sequence tract; C2′ second 3′ common sequence tract (identical to C2); RC1: sequence reverse complementary to C1; RC2: sequence reverse complementary to C2; black lines: first and second sequence.

FIG. 4 shows heteroduplex peaks from wild type and 1 bp insertion/deletion mutant in plant (a, b and c), bacteria (d) and human (c) DNA fragments detected by prePIRMA. Arrow heads indicate. Star indicates homoduplex peak.

FIG. 5 shows the detection of 0 to 7 bp gap sequences of RDP1 with HMA by using 130 bp (b) and 300 bp (c) of PCR fragments.

FIG. 6 shows the detection of 0 to 7 bp gap sequences of DML1 with HMA by using 153 bp (b) and 300 bp (c) of PCR fragments.

FIG. 7 shows Detection of 0 to 7 bp gap sequences with HMA. (a) RDP1, (b) DML1. Red arrowheads indicate heteroduplex peaks. Star indicates homoduplex peak.

FIG. 8 shows that a probe of PRIMA does not work when the mutation position is close to edge of the DNA fragment (a,b,c) and probe length was not affected to heteroduplex peak (c). No heteroduplex peak was formed using primer pair (red arrows) close to mutation position (a and b). On the other hand, heteroduplex peaks were produced when mutation position is close to middle of DNA fragment. (green arrows, a and c) Note that no big difference was detected by using 40 mer probe and 80 mer probe (c). Star indicates homoduplex peak.

FIG. 9 shows the electrophoresis patterns from 10 bp deletion to 10 bp insertion sequences with PRIMA. A. RDP1 sequences. 225 bp sequence of RDP1 was used this analysis. Red arrows indicate primer regions and blue arrow indicates probe region. Used 10 bp deletion to 10 bp insertion sequences are shown below. B and C. Poly acrylamide gel images with PRIMA. Red stars indicate homoduplex peaks. Red and blue arrowheads indicate heteroduplex from wild type and mutant sequences, respectively. Electrophoresis patterns from 10 bp deletion (del) to wildtype are shown in B and from wild type to 10 bp insertion (ins) are shown in C. D and E. MultiNA images with PRIMA. Red stars indicate homoduplex peaks. Red and blue arrowheads indicate heteroduplex from wild type and mutant sequences, respectively. Electrophoresis patterns from 10 bp deletion (del) to wildtype are shown in D and from wild type to 10 bp insertion (ins) are shown in E.

FIG. 10 shows genotyping by using HMA, prePRIMA and PRIMA. (a) Workflow of HMA for genotyping. HMA needs 2 times of analysis. 1^{st} analysis; sample is re-annealed only with sample itself. When heteroduplex peaks are formed, this sample is heterozygous. No heteroduplex peak indicate this sample is wild type or mutant homozygous. 2^{nd} analysis; sample is re-annealed with wild type sample. When heteroduplex peaks are produced, this sample is mutant homozygous and if not, this is wild type homozygous. (b) Workflow of PRIMA and prePRIMA for genotyping. Only single analysis needs to detect genotype. Examples for genotyping are shown in (c) for prePRIMA and (d) for PRIMA. Star indicates homoduplex peak.

FIG. 11 shows genotyping with PRIMA using a 225 bp PCR product of the RDP1 gene and a 40mer probe with a deletion of 5 nucleotides.

FIG. 12 shows the detection of 1 bp difference from plants (A, B, E, F), human (C and G) and bacteria (D and H) many sequences with PRIMA. Electropherogram patterns were obtained by MultiNA (A-D) and gel images were obtained by polyacrylamide gel electrophoresis (E-H).

FIG. 13 shows that PRIMA is possible to distinguish type of base (A,T,G and C). To test whether PRIMA is further usable for SNP typing, PRIMA was performed with base-edited sequences (Fig. A) using 2 different probes (Fig. A, B and C). In Fig. B, nucleotide NG and T/C is distinguishable because they produce different heteroduplex peaks. In Fig. C, NG, T and C could be distinguished. These results suggest that PRIMA has the possibility to expand its usage for SNP typing. Fig. A; red arrows indicate primers, green and blue arrows indicate probes using Fig. B (green) and Fig. C (blue). Base-editing point is shown in black arrow. Fig. B, C SNP typing with PRIMA using 5531 probe (B) and 5428 probe(C). Black, green, red and blue arrowheads indicate heteroduplex peaks from A, T, G and C, respectively.

FIG. 14 shows the detection 1 bp difference with PRIMA. A. Gene construction of RDP1. Red arrows indicate primer regions and blue arrow indicates probe region. Red square shows mutation position. B. Detection of heteroduplex peak using MultiNA, Red star indicates homoduplex peaks and blue arrowheads indicate heteroduplex peaks. C. Detection of heteroduplex peak using poly acrylamide gel. Red star indicates homoduplex peaks and blue arrowheads indicate heteroduplex peaks. Marker (M) sizes are shown at left side. Different size of heteroduplex peaks were detected from 1 ins, wild type and 1del sequence with MultiNA and PAGE.

FIG. 15 shows the protocol for PRIMA.

FIG. 16 shows an alternative approach for describing the variable sequence tract V.

FIG. 17 shows a comparison of deletion or insertion probe with 1-bp indel mutants. Expected bulge structures showed that a deletion probe is simpler and has a more distinguishable bulge than the insertion probe, even though the mutation position is shifted by a few-bp (FIG. 17). Therefore, rather than using a 5-bp insertion probe, preferably a 5-bp deletion probe may be used so that the bulge size would be different from the WT, even when the 1-bp indel position is a few-bp away because exact indel positions induced by a single CRISPR experiment are known to be variable within the range of a few-bp (Nishida et al. Science 353, (2016)). Expected bulge structures are shown in wild type and 1-bp indel mutants which have 5-bp position-shifted mutation (−2 to +3). Deletion probe produces simple and distinguishable bulge structure from all insertion (a) and deletion (b) mutants. On the other hand, insertion probe produces simple bulge structure only “+1” and “+2” from deletion series (a) and “+1” from insertion series (b). Upper strand of heteroduplex figure comes from sample DNA. Lower strand of heteroduplex figure comes from probe DNA. Arrowheads indicate +1 position. Grey line indicates null nucleotide. Purple line indicates 5-bp insertion nucleotide in insertion probe. Red line indicates 1-bp insertion nucleotide in insertion series. Red squares indicate when a different bulge structure compared to the wild type is expected.

### SEQUENCES

The following sequences appear in the Figures:

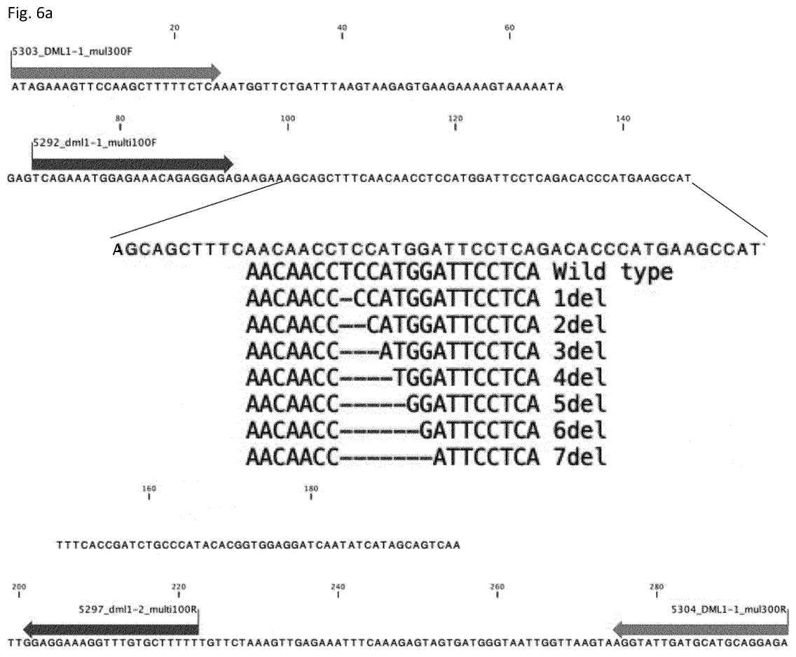

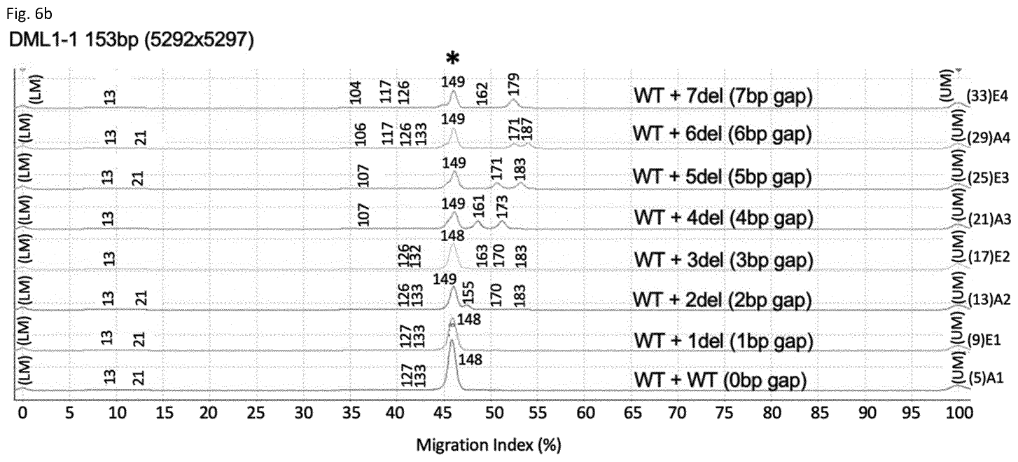

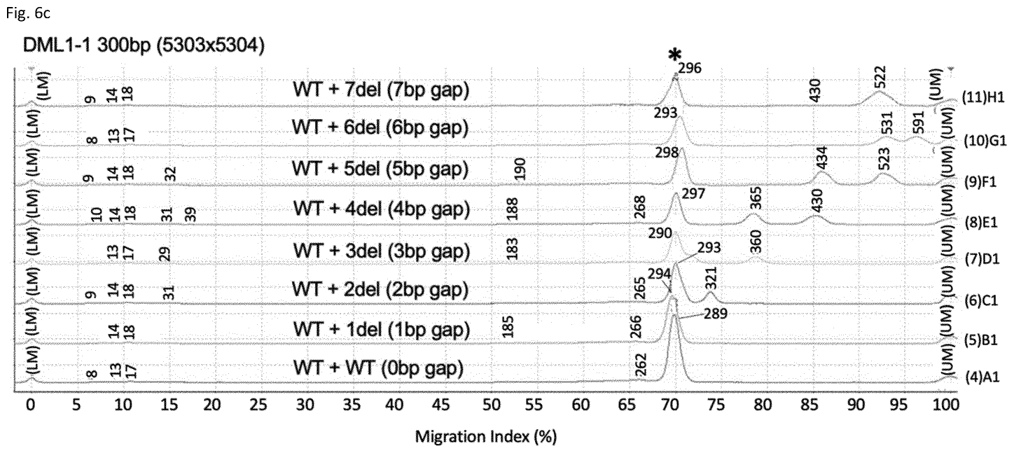

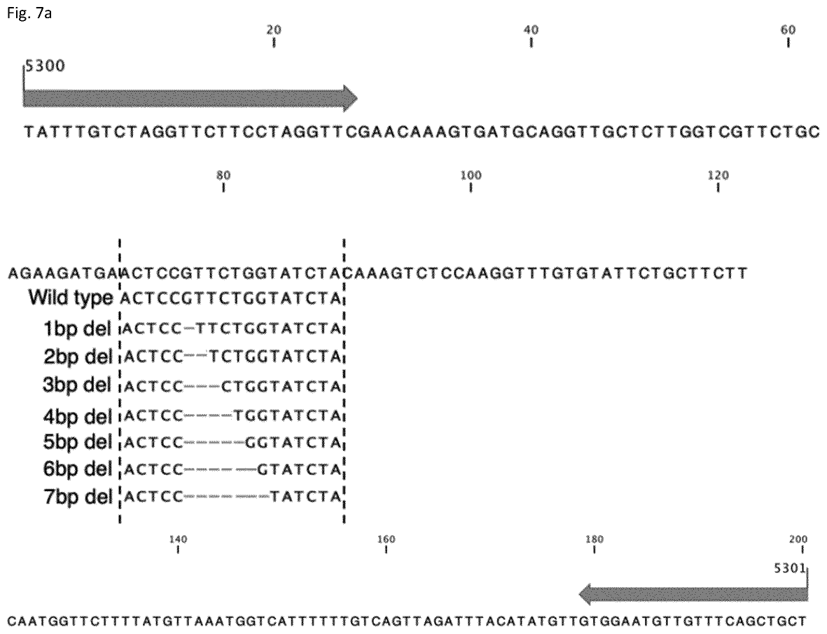

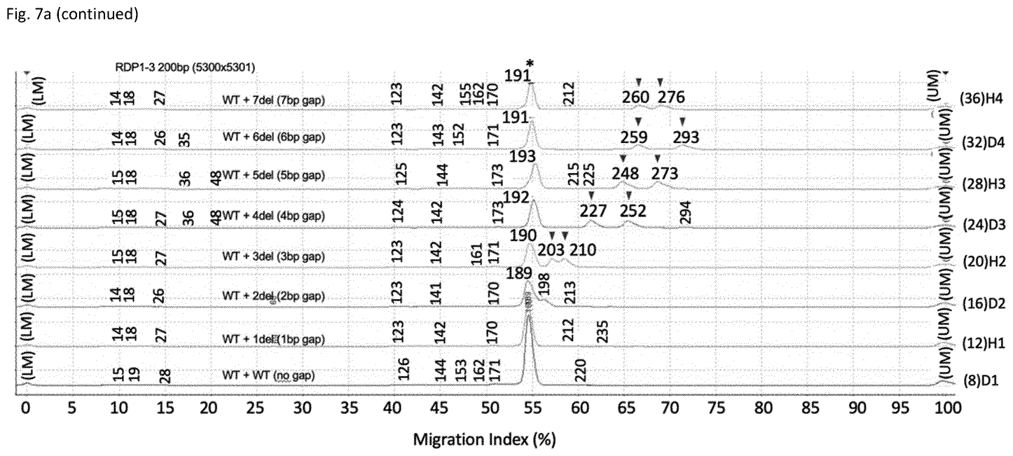

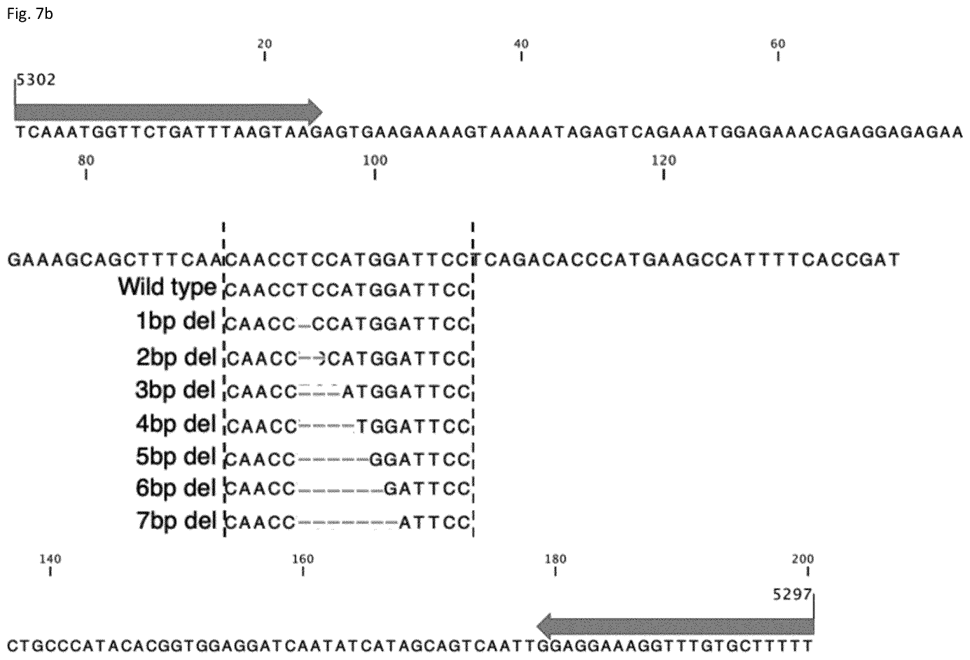

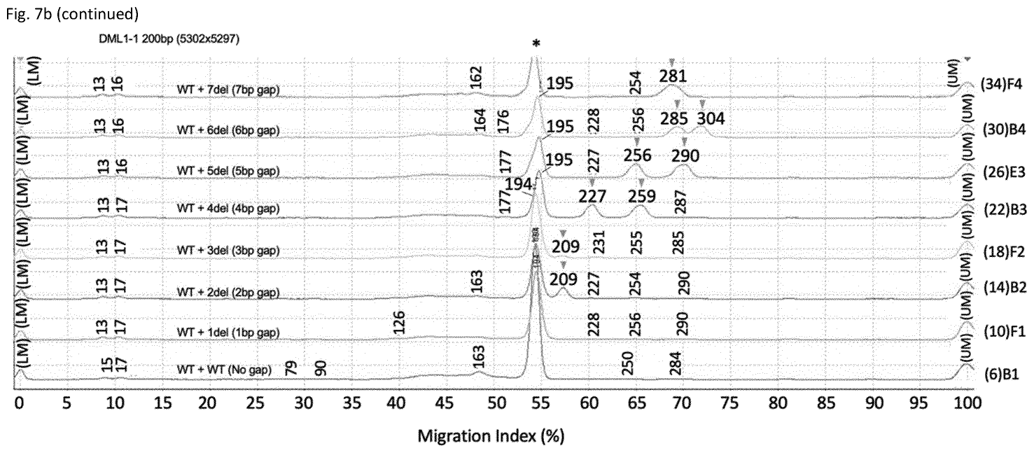

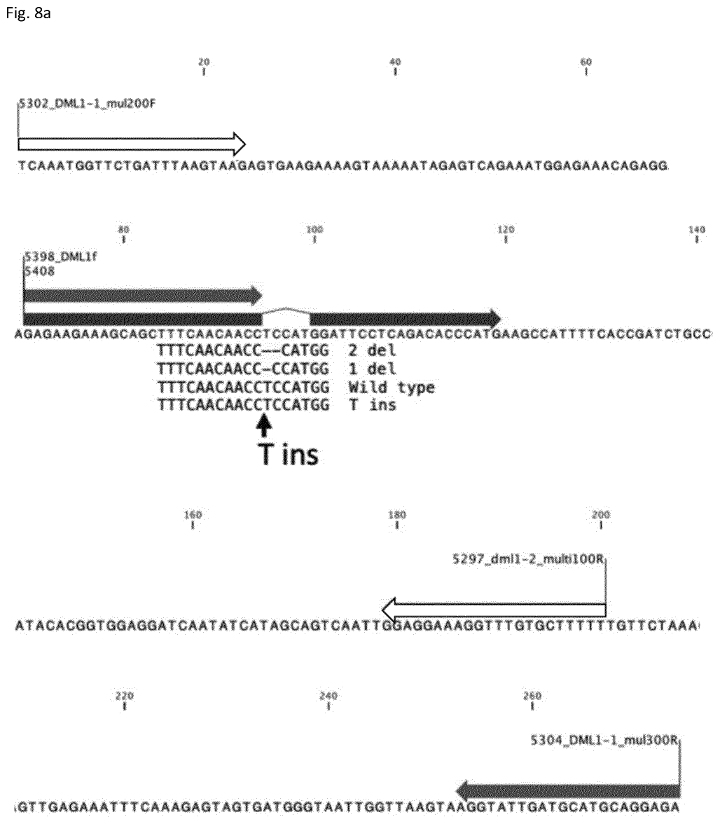

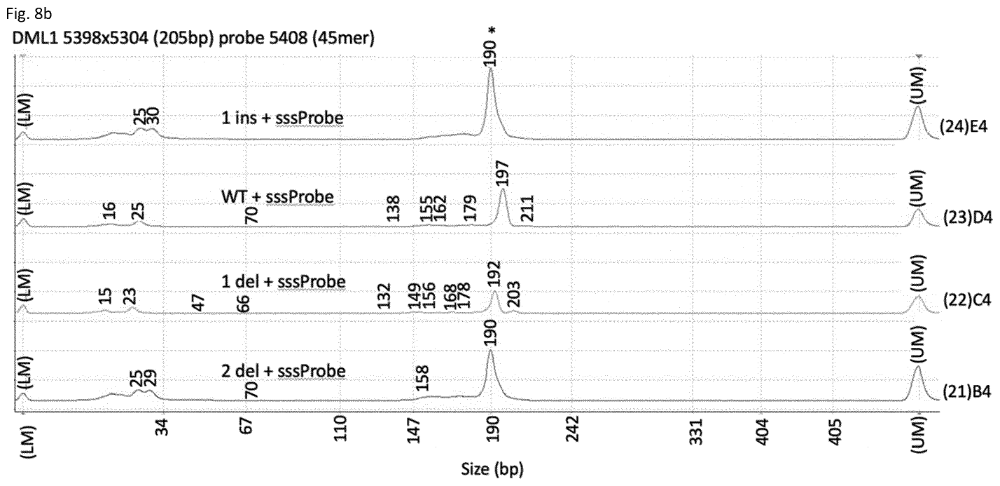

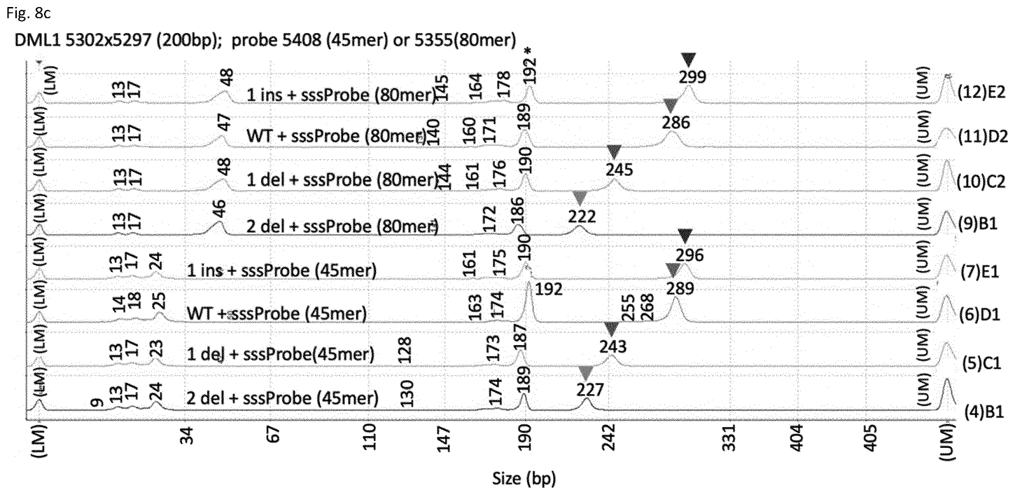

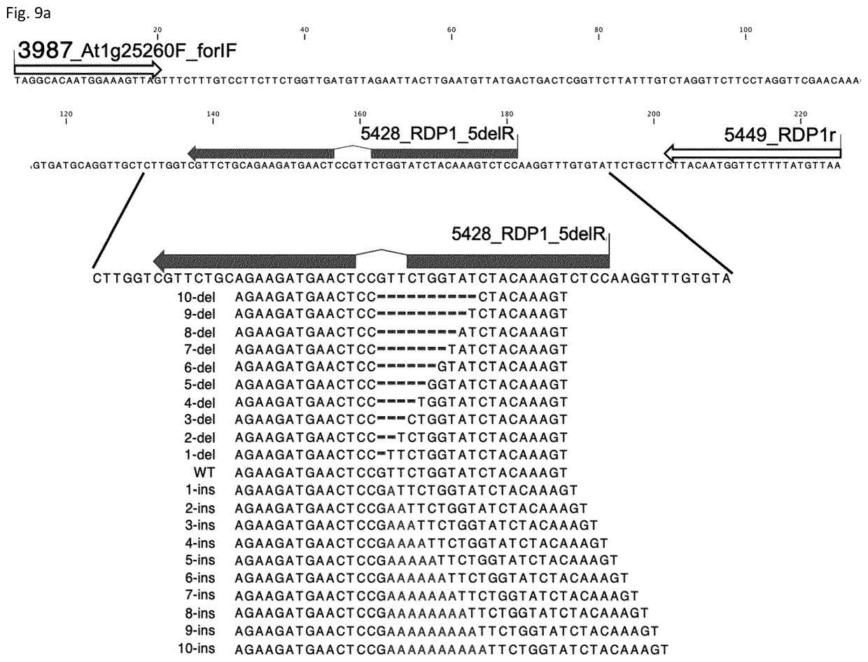

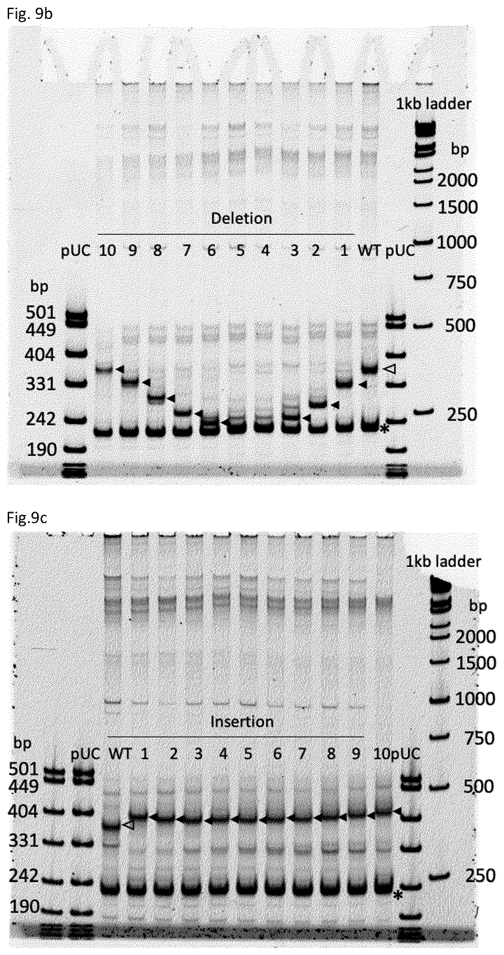

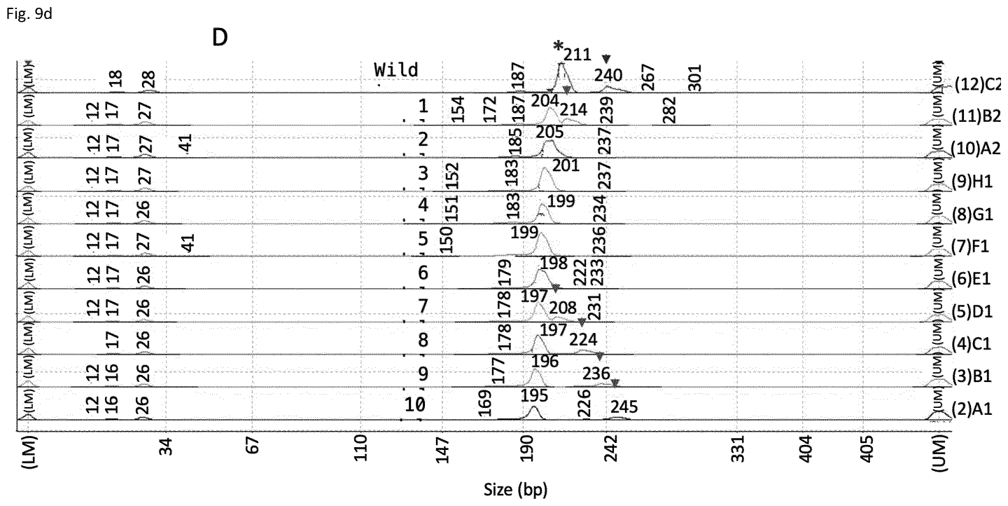

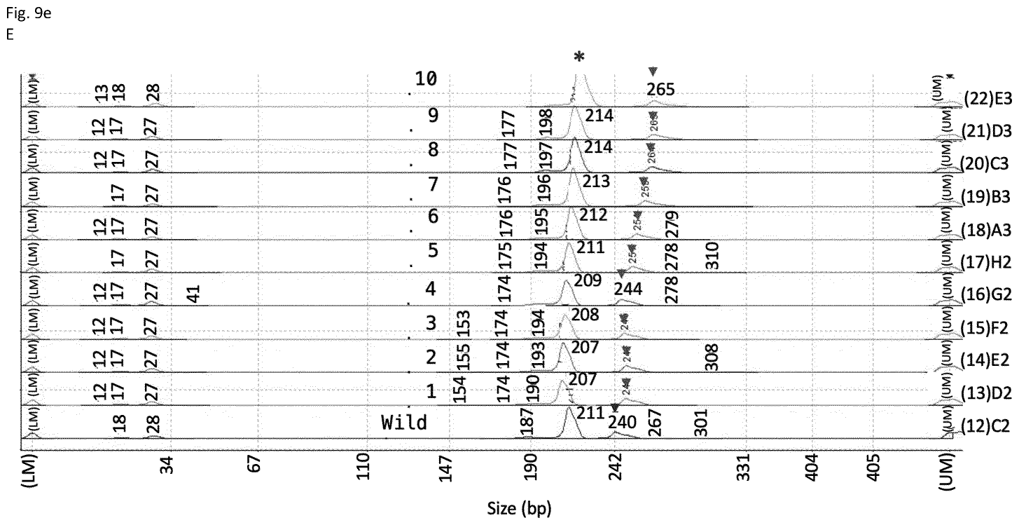

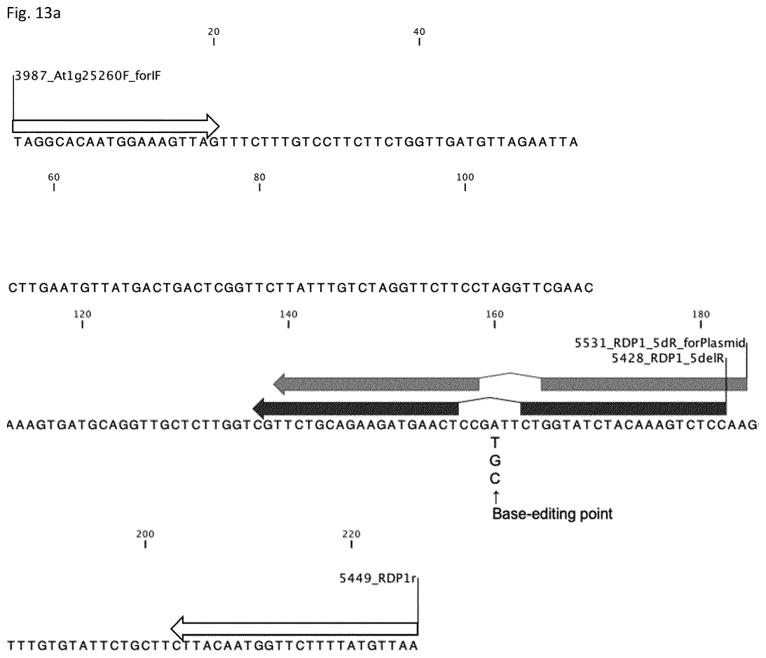

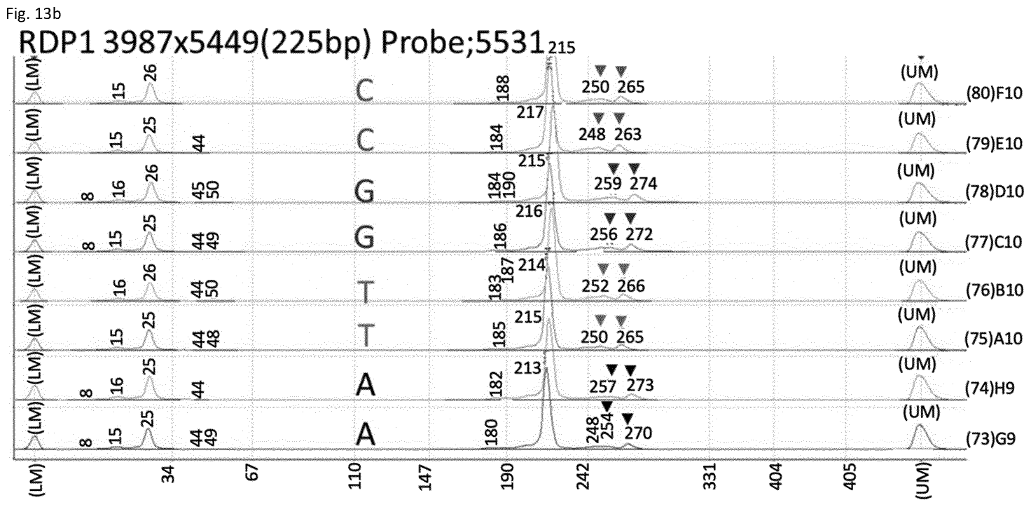

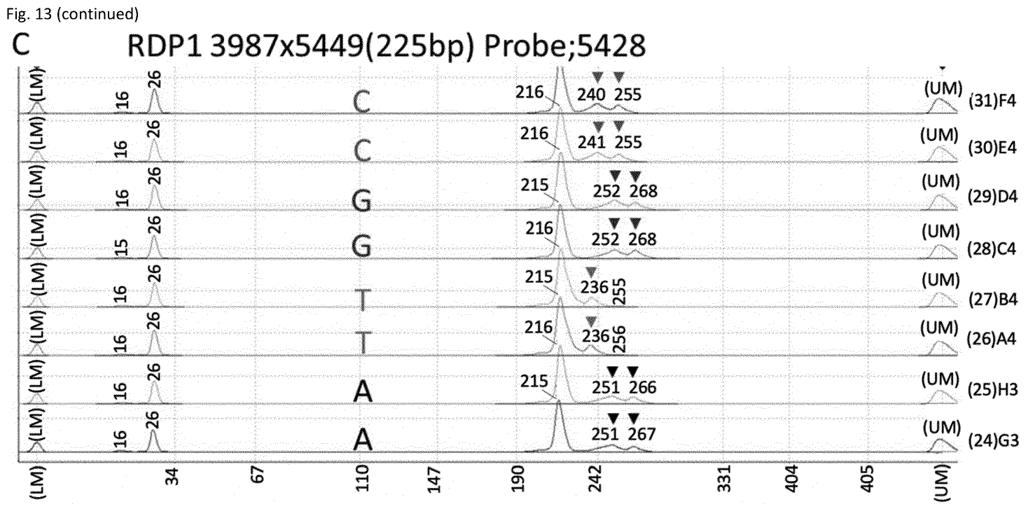

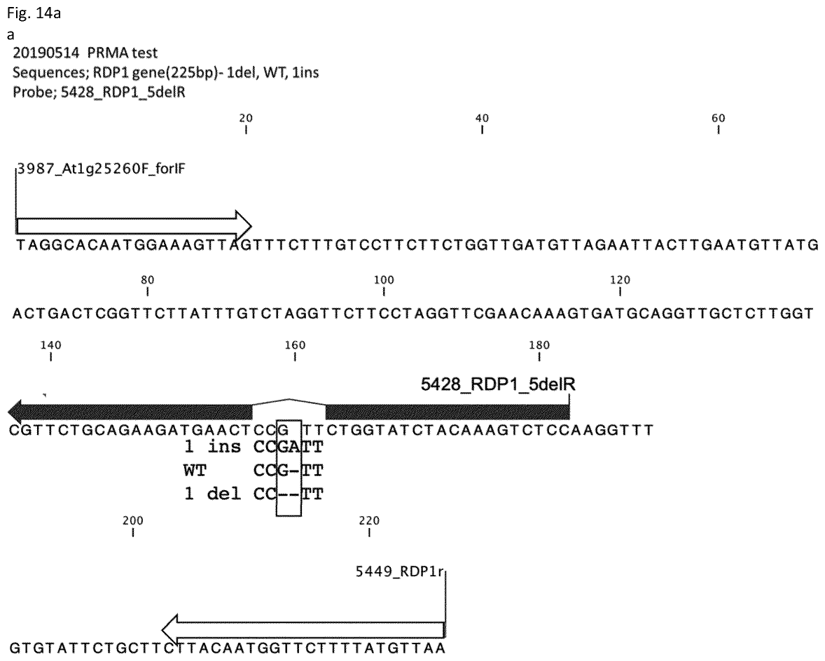

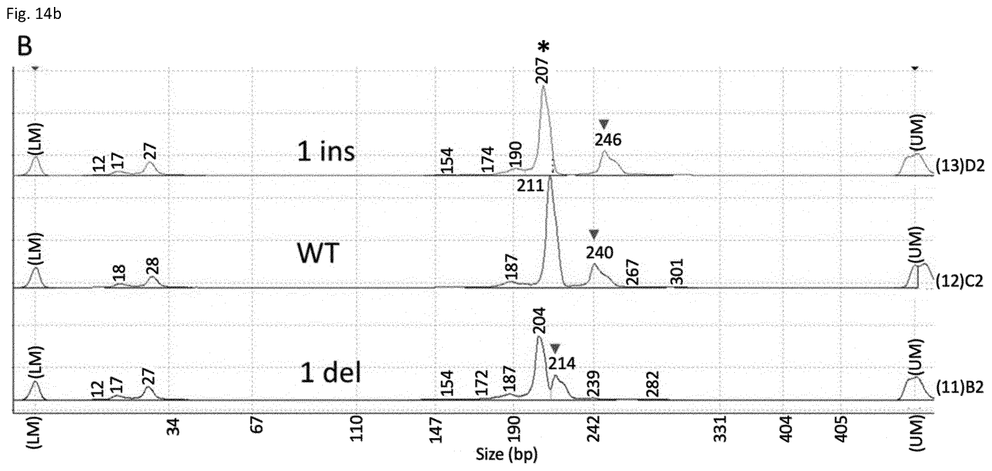

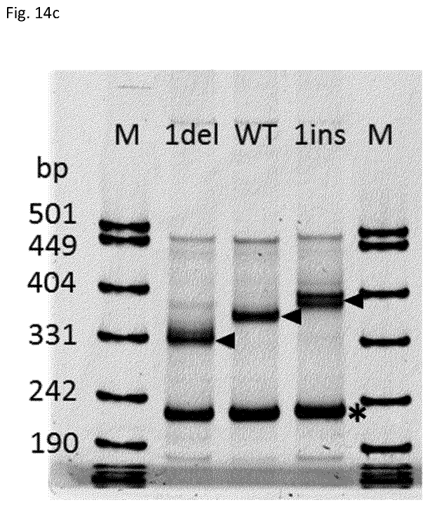

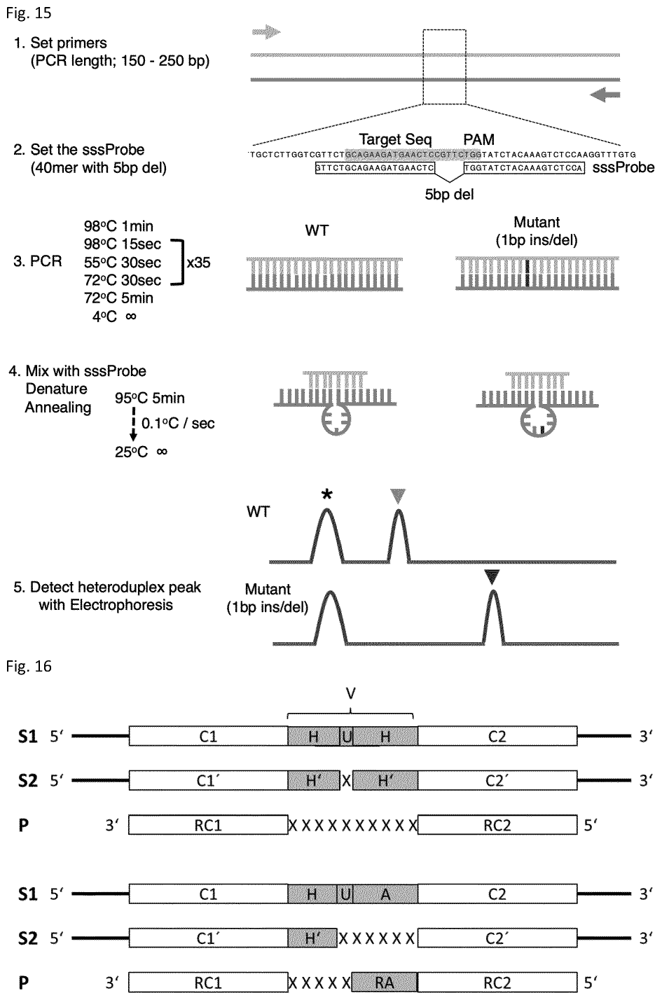

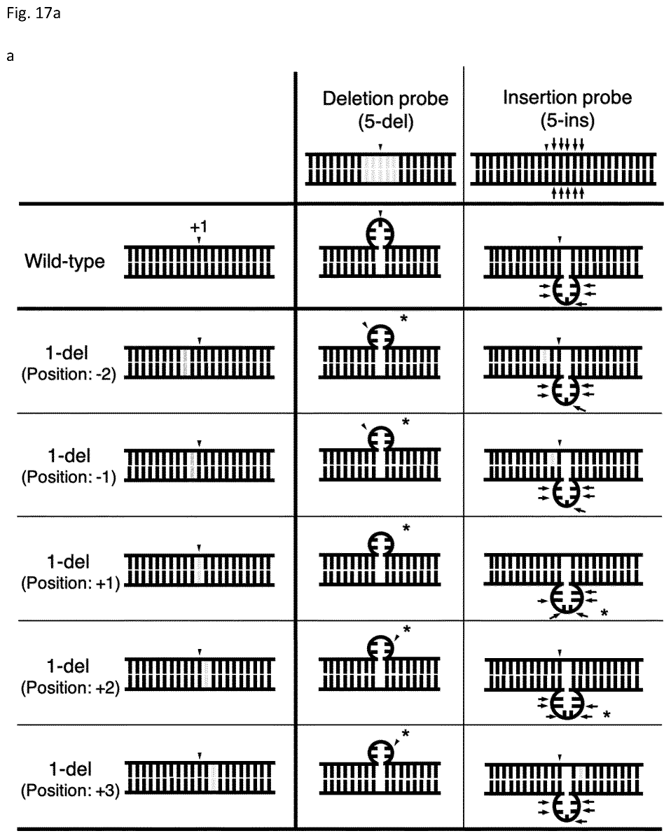

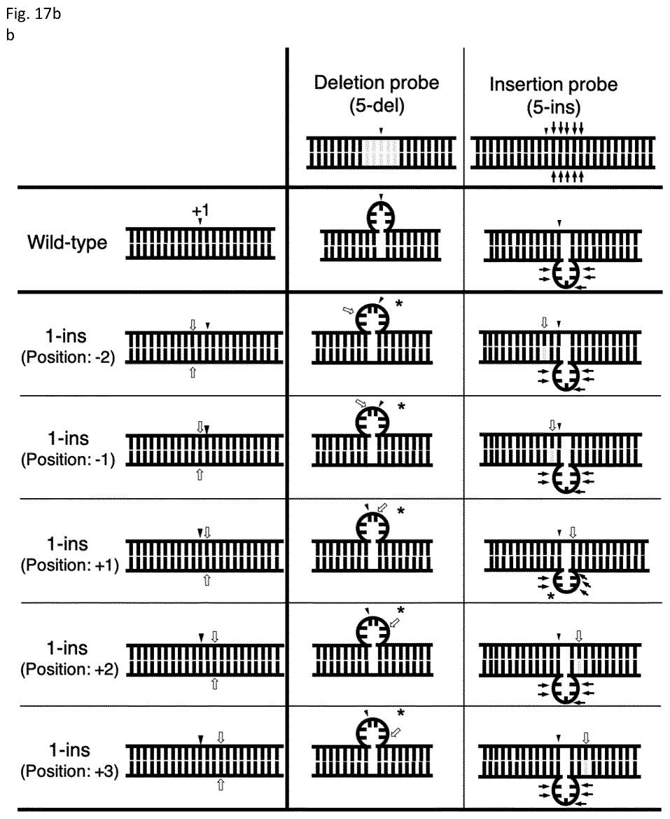

FIG. 5a
RDP1_
(SEQ ID NO: 001)
CTGCAGAAGATGAACTCCGTTCTGGTATCTACAAAGTCTCCAAGGTTT
Wild type
(SEQ ID NO: 002)
GAACTCCGTTCTGGTATCTAC
1 del
(SEQ ID NO: 003)
GAACTCC TTCTGGTATCTAC
2 del
(SEQ ID NO: 004)
GAACTCC --TCTGGTATCTAC
3 del
(SEQ ID NO: 005)
GAACTCC- CTGGTATCTAC
4 del
(SEQ ID NO: 006)
GAACTCC- TGGTATCTAC
5 del
(SEQ ID NO: 007)
GAACTCC- GGTATCTAC
6 del
(SEQ ID NO: 008)
GAACTCC- GTATCTAC
7 del
(SEQ ID NO: 009)
GAACTCC- TATCTAC
FIG. 6a
DML1_
(SEQ ID NO: 010)
AGCAGCTTTCAACAACCTCCATGGATTCCTCAGAGACCCATGAAGCCAT
Wild type
(SEQ ID NO: 011)
AACAACCTCCATGGATTCCTCA
1 del
(SEQ ID NO: 012)
AACAACC-CCATGGATTCCTCA
2 del
(SEQ ID NO: 013)
AACAACC CATGGATTCCTCA
3 del
(SEQ ID NO: 014)
AACAACC ATGGATTCCTCA
4 del
(SEQ ID NO: 015)
AACAACC TGGATTCCTCA
5 del
(SEQ ID NO: 016)
AACAACC -GGATTCCTCA
6 del
(SEQ ID NO: 017)
AACAACC GATTCCTCA
7 del
(SEQ ID NO: 018)
AACAACC---ATTCCTCA
FIG. 7a
RDP1_
Wild type
(SEQ ID NO: 019)
ACTCCGTTCTGGTATCTA
1 bp del
(SEQ ID NO: 020)
ACTCC-TTCTGGTATCTA
2 bp del
(SEQ ID NO: 021)
ACTCC--TCTGGTATCTA
3 bp del
(SEQ ID NO: 021)
ACTCC---CTGGTATCTA
4 bp del
(SEQ ID NO: 022)
ACTCC----TGGTATCTA
5 bp del
(SEQ ID NO: 023)
ACTCC-----GGTATCTA
6 bp del
(SEQ ID NO: 024)
ACTCC------GTATCTA
7 bp del
(SEQ ID NO: 025)
ACTCC-------TATCTA
FIG. 7b
DML1_
Wild type
(SEQ ID NO: 026)
CAACCTCCATGGATTCC
1 by del :
(SEQ ID NO: 027)
CAACC CCATGGATTCC
2 bp del :
(SEQ ID NO: 028)
CAACC CATGGATTCC
3 bp del :
(SEQ ID NO: 029)
CAACC ATGGATTCC
4 bp del :
(SEQ ID NO: 030)
CAACC TGGATTCC
5 bp del :
(SEQ ID NO: 031)
CAACC GGATTCC
6 bp del
(SEQ ID NO: 032)
CAACC GATTCC
7 bp del
(SEQ ID NO: 033)
CAACC ATTCC
FIG. 8a
Not_
2 del
(SEQ ID NO: 034)
TTTCAACAACC--CATGG
1 del
(SEQ ID NO: 035)
TTTCAACAACC-CCATGG
Wildtype
(SEQ ID NO: 036)
TTTCAACAACCTCCATGG
T ins
(SEQ ID NO: 037)
TTTCAACAACCTCCATGG
FIG. 9a
DNA fragment with deletion
(SEQ ID NO: 038)
...AGAAGATGAACTCC----------CTACAAAGT...
(SEQ ID NO: 039)
...AGAAGATGAACTCC---------TCTACAAAGT...
(SEQ ID NO: 040)
...AGAAGATGAACTCC--------ATCTACAAAGT...
(SEQ ID NO: 041)
...AGAAGATGAACTCC-------TATCTACAAAGT...
(SEQ ID NO: 042)
...AGAAGATGAACTCC------GTATCTACAAAGT...
(SEQ ID NO: 043)
...AGAAGATGAACTCC-----GGTATCTACAAAGT...
(SEQ ID NO: 044)
...AGAAGATGAACTCC----TGGTATCTACAAAGT...
(SEQ ID NO: 045)
...AGAAGATGAACTCC---CTGGTATCTACAAAGT...
(SEQ ID NO: 046)
...AGAAGATGAACTCC--TCTGGTATCTACAAAGT...
(SEQ ID NO: 047)
...AGAAGATGAACTCC-TTCTGGTATCTACAAAGT...
wildtype
(SEQ ID NO: 048)
...AGAAGATGAACTCCGTTCTGGTATCTACAAAGT...
DNA fragment with insertion
(SEQ ID NO: 049)
(SEQ ID NO: 001)...AGAAGATGAACTCCGATTCTGGTATCTACAA
AGT...
(SEQ ID NO: 050)
...AGAAGATGAACTCCGAATTCTGGTATCTACAAAGT...
(SEQ ID NO: 051)
...AGAAGATGAACTCCGAAATTCTGGTATCTACAAAGT...
(SEQ ID NO: 052)
...AGAAGATGAACTCCGAAAATTCTGGTATCTACAAAGT...
(SEQ ID NO: 053)
...AGAAGATGAACTCCGAAAAATTCTGGTATCTACAAAGT...
(SEQ ID NO: 054)
...AGAAGATGAACTCCGAAAAAATTCTGGTATCTACAAAGT...
(SEQ ID NO: 055)
..AGAAGATGAACTCCGAAAAAAATTCTGGTATCTACAAAGT...
(SEQ ID NO: 056)
...AGAAGATGAACTCCGAAAAAAAATTCTGGTATCTACAAAGT...
(SEQ ID NO: 057)
...AGAAGATGAACTCCGAAAAAAAAATTCTGGTATCTACAAAGT...
(SEQ ID NO: 058)
...AGAAGATGAACTCCGAAAAAAAAAATTCTGGTATCTACAAAGT..
FIG. 13
(SEQ ID NO: 059)
CTCTTGGTCGTTCTGCAGAAGATGAACTCCGATTCTGGTATCTACAAAGT
CTCCAAGGTTT
FIG. 14
1insertion (1ins)
(SEQ ID NO: 060)
GGTCGTTCTGCAGAAGATGAACTCCGATTCTGGTATCTACAAAGTCTCCA
AGGTTTGTGTA
Wild type (WT)
(SEQ ID NO: 061)
GGTCGTTCTGCAGAAGATGAACTCCG_TTCTGGTATCTACAAAGTCTCCA
AGGTTTGTGTA
1bpdeletion (idel)
(SEQ ID NO: 062)
GGTCGTTCTGCAGAAGATGAACTCCTTCTGGTATCTACAAAGTCTCCAAG
GTTTGTGTA
FIG. 15
Targetseq
(SEQ ID NO: 063)
GCAGAAGATGAACTCCGTTCTGG
5BP DEL
(SEQ ID NO: 064)
GTTCTGCAGAAGATGAACTC
(SEQ ID NO: 065)
TGGTATCTACAAAGTCTCAA

### EXAMPLES

### Example 1: The Pattern and the Resolution of Heteroduplex Mobility Assay (HMA)

The inventors tested the band patterns of traditional HMA with MultiNA, Microchip Electrophoresis System from SHIMADZU. A wild type sequence and mutant sequences carrying different lengths of deletions, i.e. 0 bp (wild type) to 7 bp deleted sequences were amplified separately by PCR. Then the PCR product from the wild type was mixed with the PCR product from mutant sequences, respectively. These mixtures are denatured and re-annealed to introduce the heteroduplex complex. If the gap is enough long, the mismatched DNA sequences can arise a bulge caused by looped out bases, resulting in mobility shift (Bhattacharyya and Lilley, 1989 NAR). Similar to the previously shown results, the inventors could not detect 1 bp difference with any heteroduplex peaks (Bhattacharyya and Lilley, 1989 NAR). The heteroduplex peak with 2 bp gap was not clear neither (Ota et al., 2013 Genes Cells, Ansai et al., 2014 Dev Growth Differ).

### Example 2: HMA with 5 bp Deletion Probe (prePRIMA)

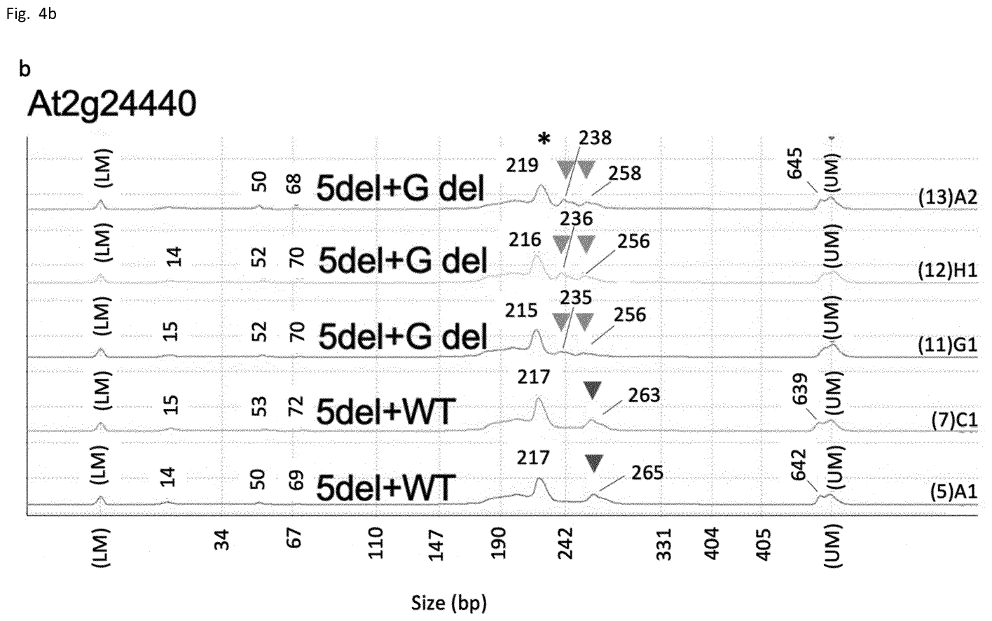

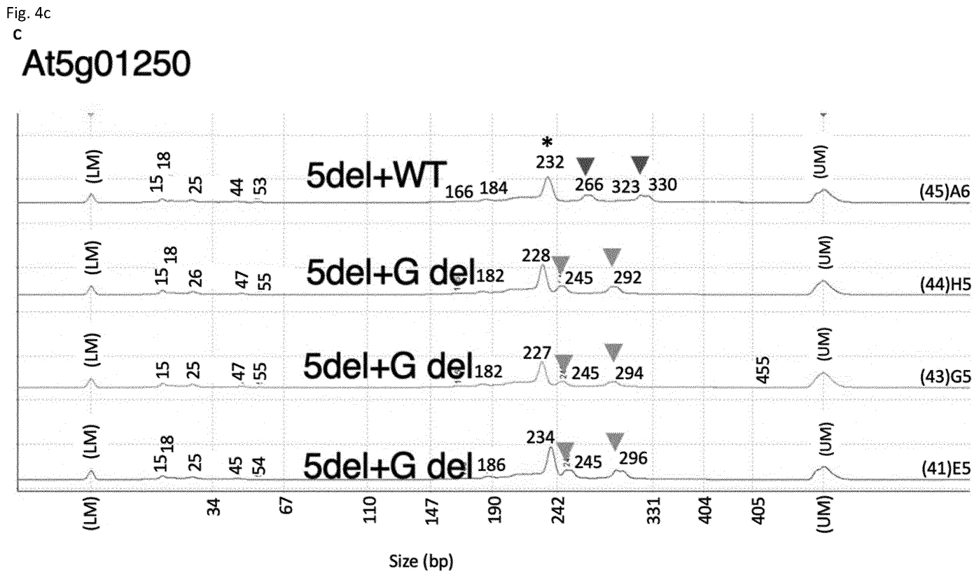

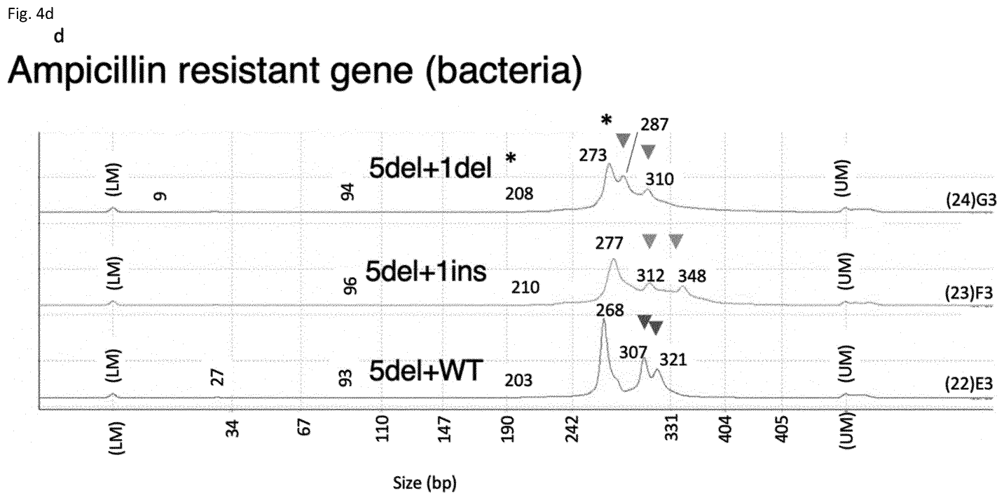

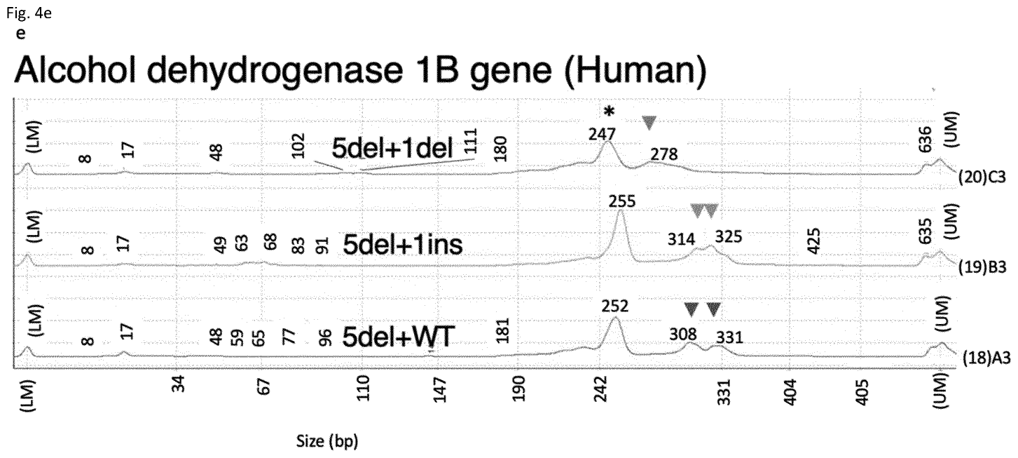

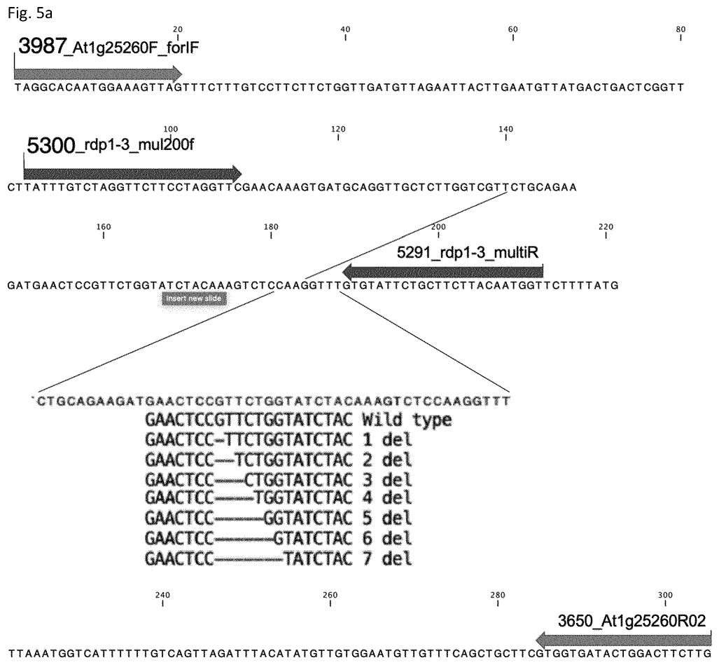

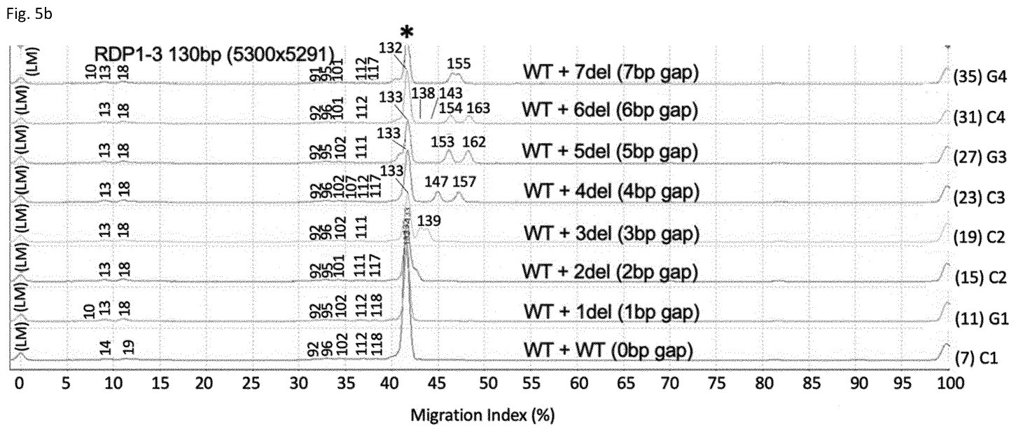

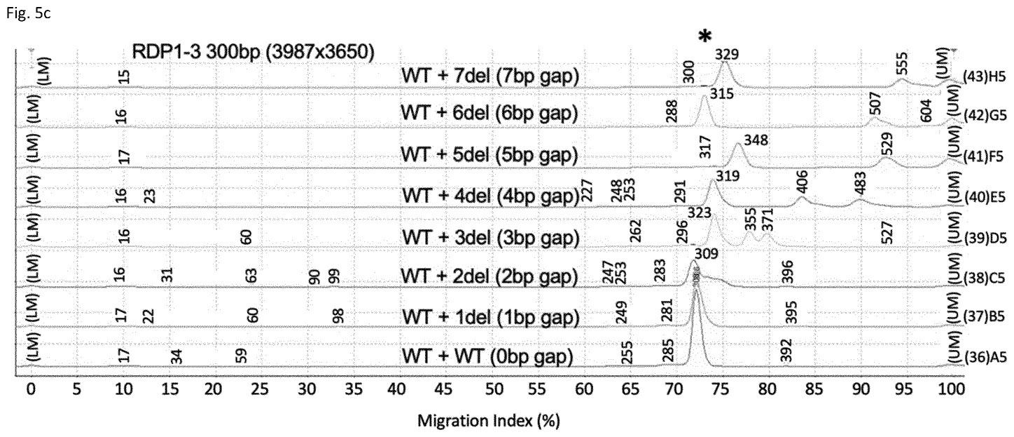

The inventors proceeded with the objective of detecting a 1 bp length difference. They tested whether it was possible to distinguish 4 bp (=1 bp deletion), 5 bp (=wild type) and 6 bp (=1 bp insertion) using 5 genes which are either from A. thaliana, bacteria or human. Indeed, the inventors clearly identified the 1 bp insertion and deletion in all cases (FIG. 4). The inventors refer to this technique as prePRIMA (precursive method of Probe-Induced HMA).

The inventors further examined the effect of PCR fragment sizes and/or different sequences (FIGS. 5 and 6). Fragment with about 200 bp size worked well to detect different heteroduplex peaks among 3 to 7 bp gap fragments (FIG. 7). While shorter fragment (i.e. 130 bp of RDP1 and 153 bp of DML1 in FIG. 5b and FIG. 6a) was not adequate to obtain clear differences. Heteroduplex peaks derived from 300 bp fragments sometimes overlapped with upper marker in our system and cannot be analyzed by using MultiNA chip 500 (FIG. 5c and FIG. 6c).

The inventors further aimed to optimize the probe design. A probe worked better when it has the gap region overlapped with the mutated site at the middle of the PCR fragment than at the edge of the PCR fragment (FIG. 8).

### Example 3: PRIMA with Short Single-Strand DNA (sssDNA) Probe

It is time-consuming to make a probe with 5 bp deletion in the middle of 200 bp PCR fragment, because it needs 2 step PCR or Cloning (Braman 2004, Springer protocols/Methods in Mol Bio1634). Otherwise, it is possible to order longer oligos but the cost becomes relatively expensive.

To overcome these obstacles, the inventors examined if a single-strand DNA (ssDNA) may enough to produce a heteroduplex with looped out bases. The results are shown in FIG. 8c. The ssDNA (80mer) was enough to discriminate the 1 bp different sequences. It was also possible to shorten this ssDNA probe to decrease the cost of oligonucleotide synthesis. The inventors found that short ssDNA (sssDNA) such as 40mer would be enough (FIG. 8c). From these findings, the inventors named this method as PRIMA (Probe-Induced Heteroduplex Mobility Assay) with sssDNA. It is also important that the sssDNA prefer to set around middle of the DNA fragment (FIG. 8).

### Example 4: Screening by PRIMA

The inventors tested PRIMA with 10 deletion to 10 insertion mutated sequences of RDP1 (FIG. 9). There are heteroduplex peaks with different sizes of deletion to insertion sequences (FIG. 9). These results suggest that PRIMA can work in mutant screening. This can be a great help to reduce the cost of time and money in the broad range of biological researchers.

### Example 5: Genotyping by PRIMA

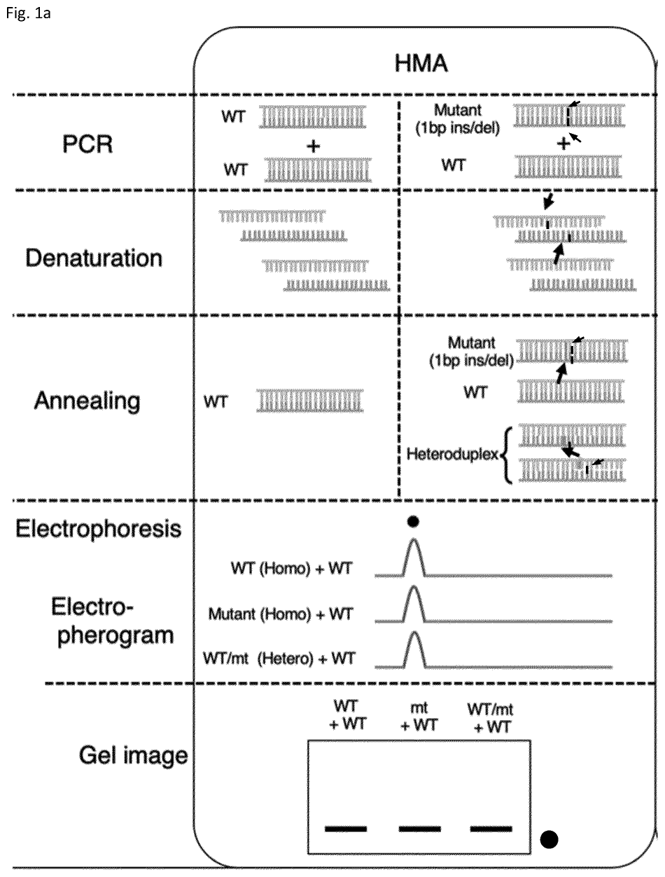

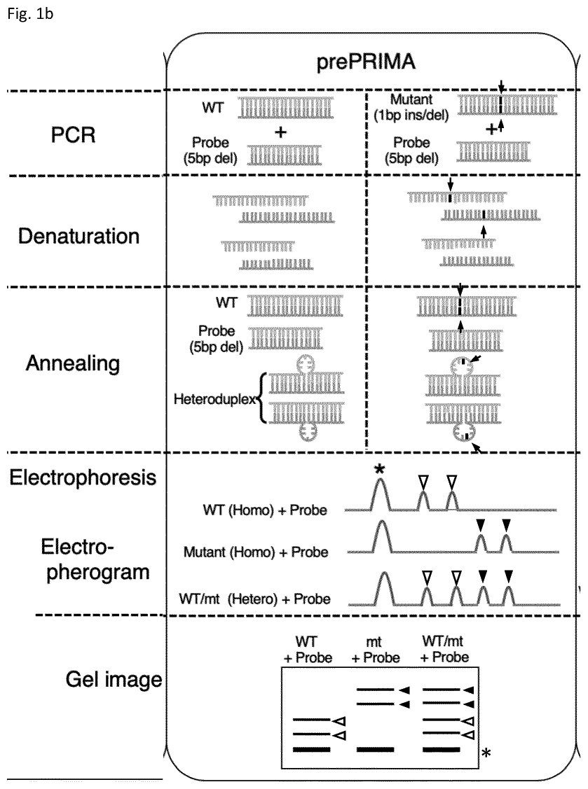

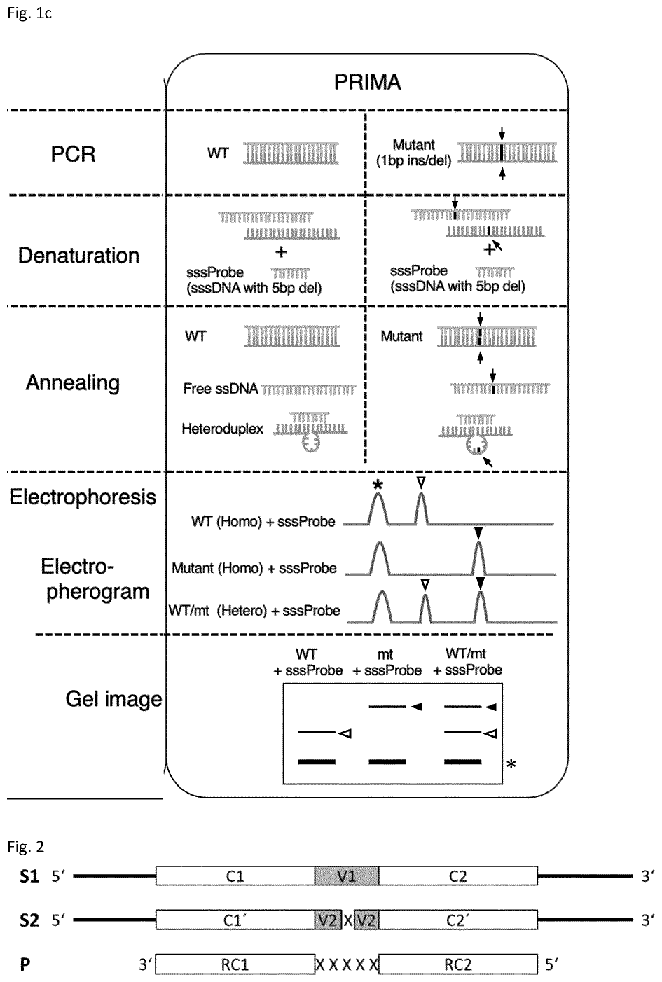

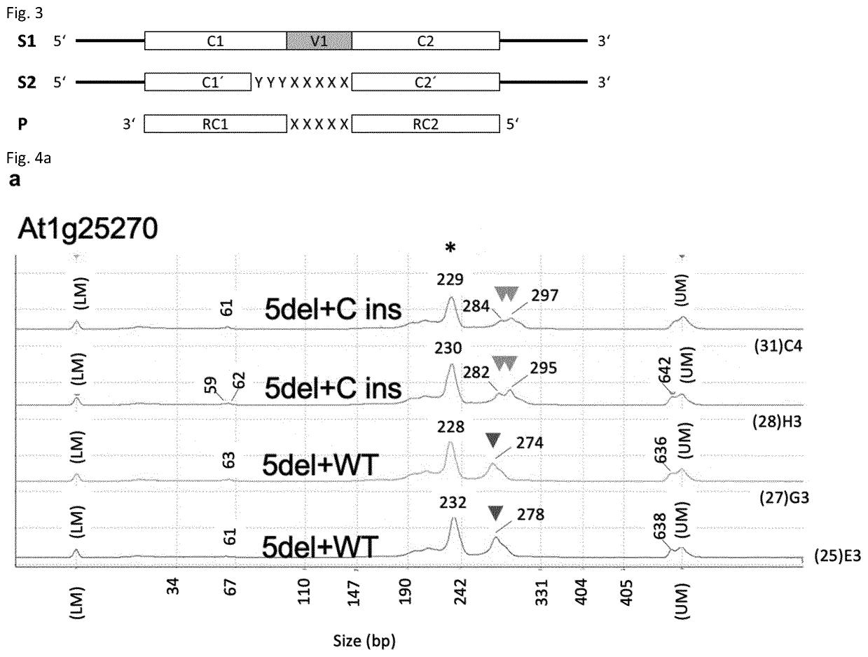

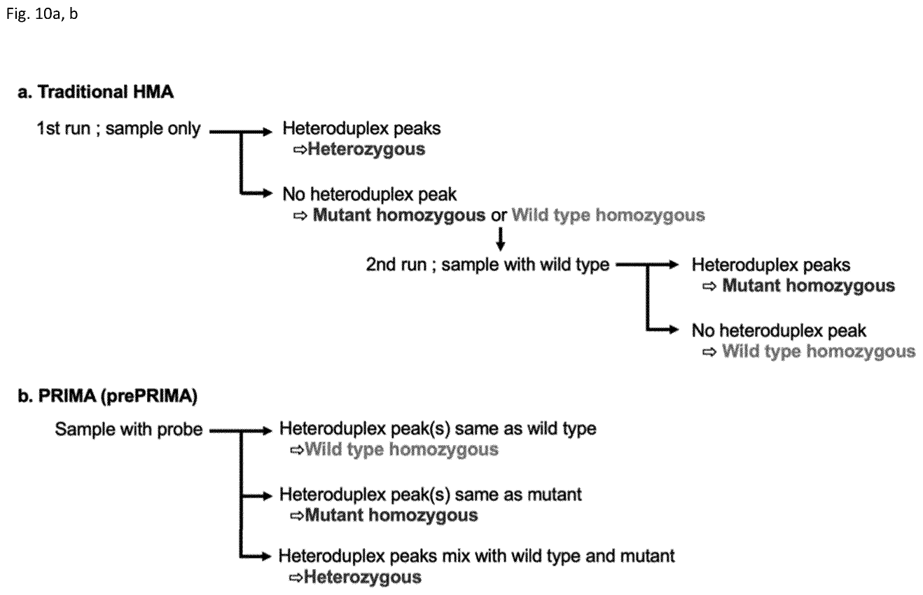

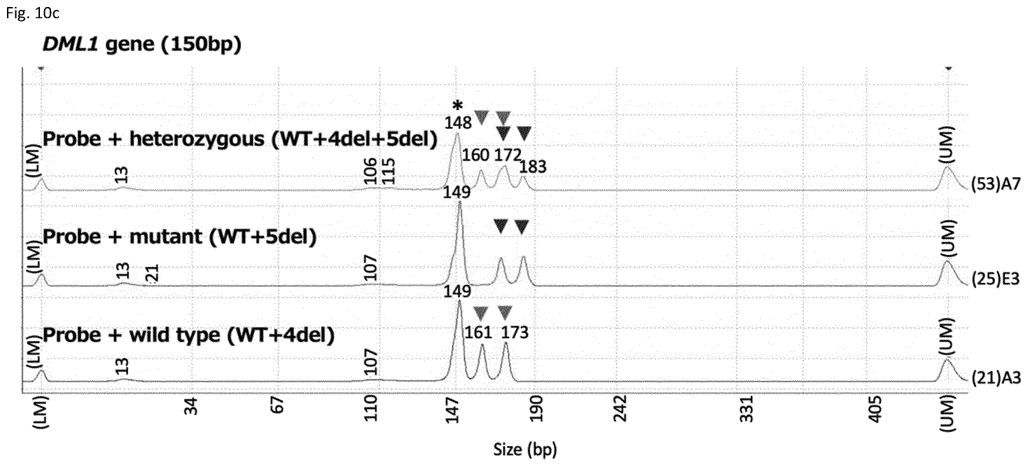

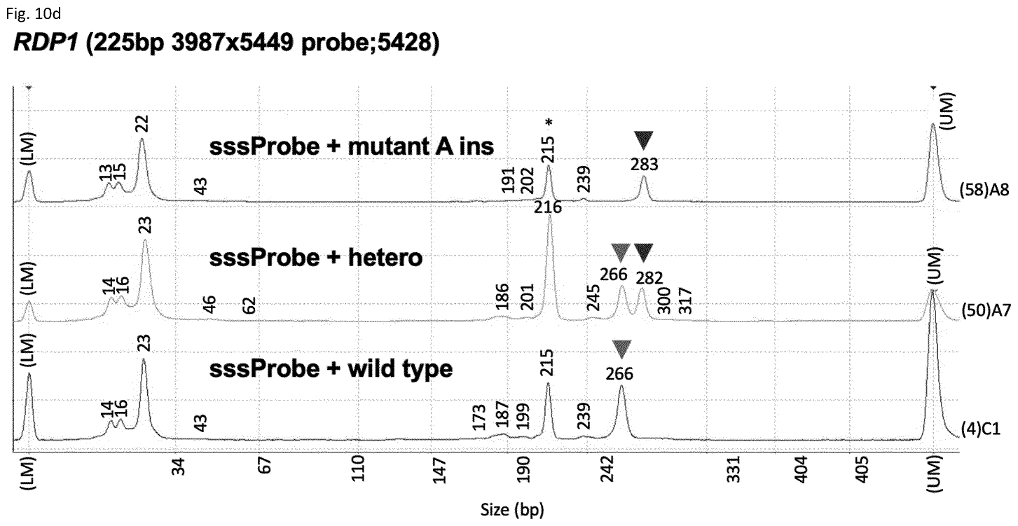

Traditional HMA has been used for genotyping, (Ansai et al., 2014 Dev Growth Differ), although, the resolution of HMA is low as we also showed above (FIG. 1). Because of this low resolution, 1 bp different heterozygous genotype cannot be distinguished. Even when a few bp difference can be detected from the mobility shift of the heteroduplex, it is often not possible to distinguish the 2 homozygous genotype (i.e. wild type and mutant) with the small difference (a few bp). Researchers run another sample set of HMA to distinguish these homozygous wild type and mutant (FIG. 10a).

It is possible to conduct the two types of runs at the same time to save time, but the researchers need to analyse twice as many as the sample number.

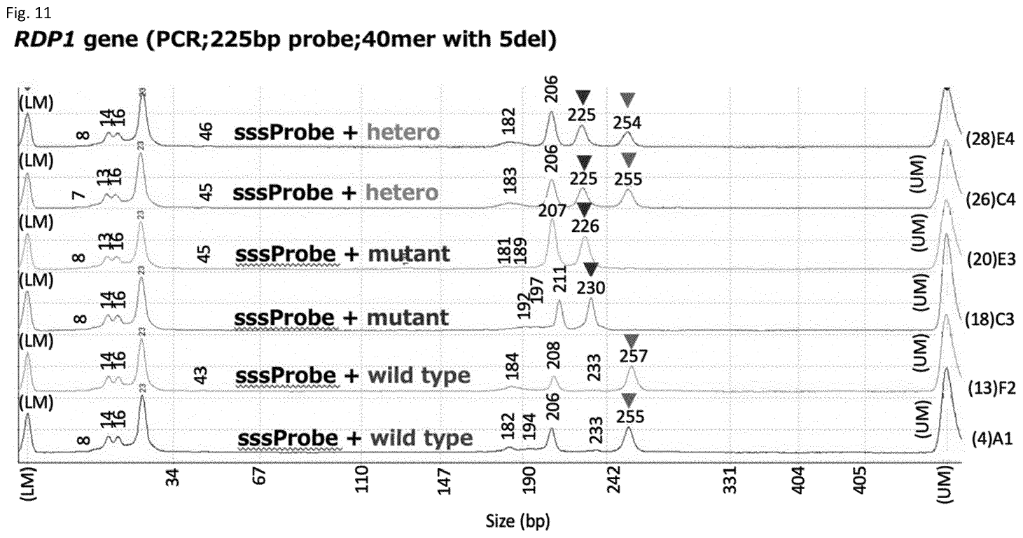

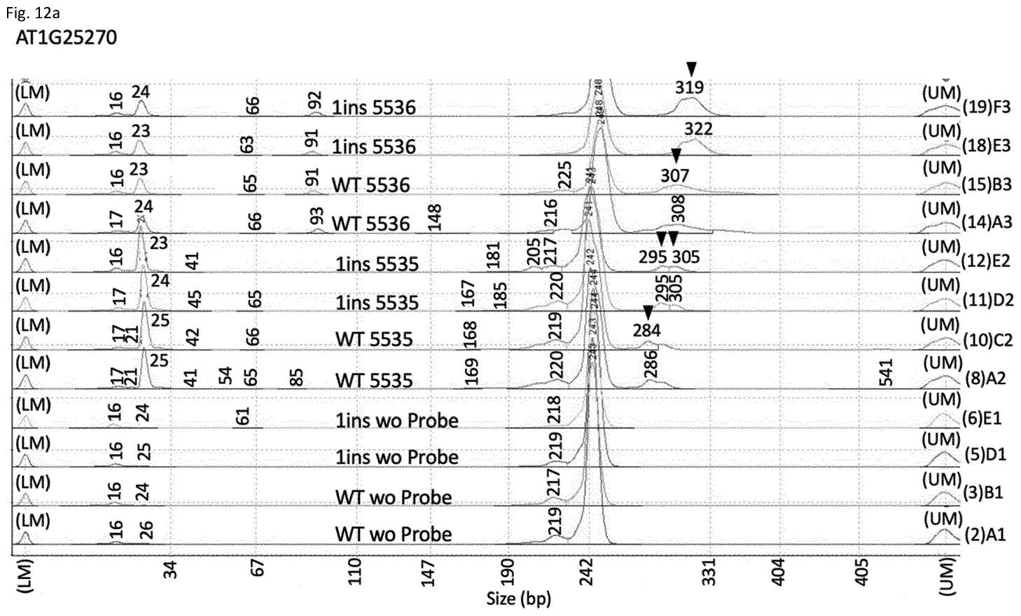

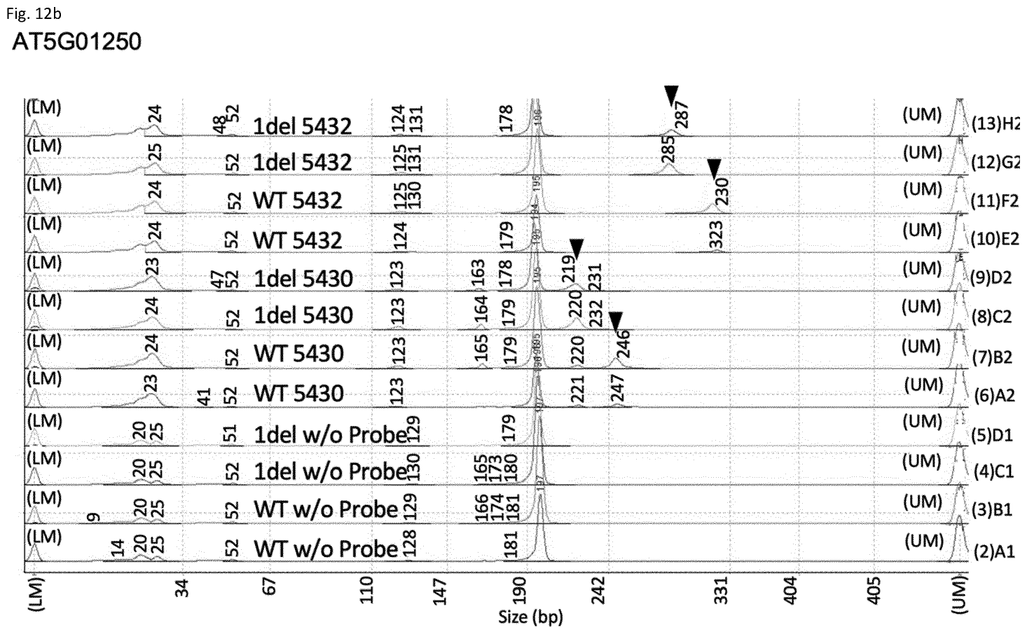

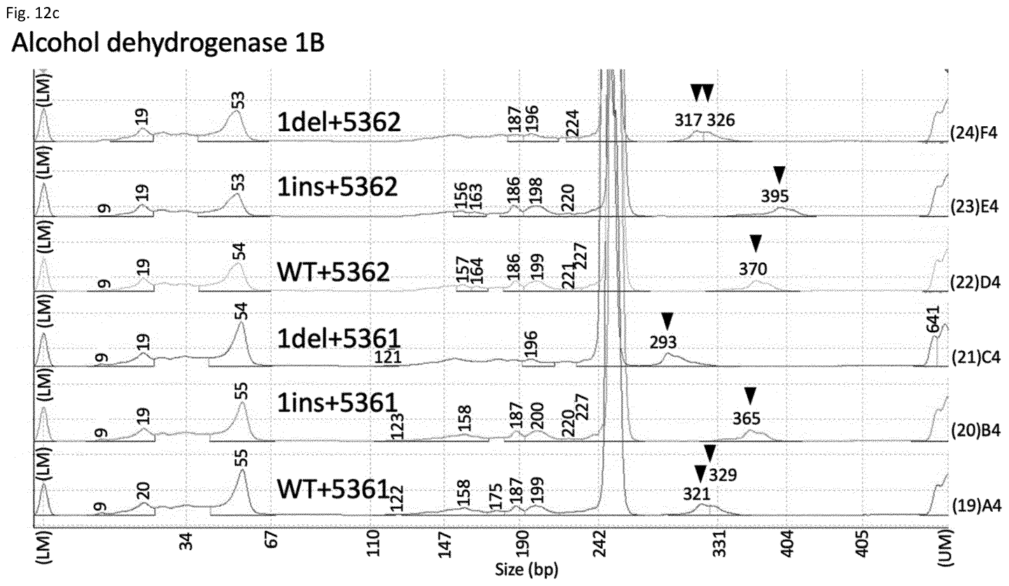

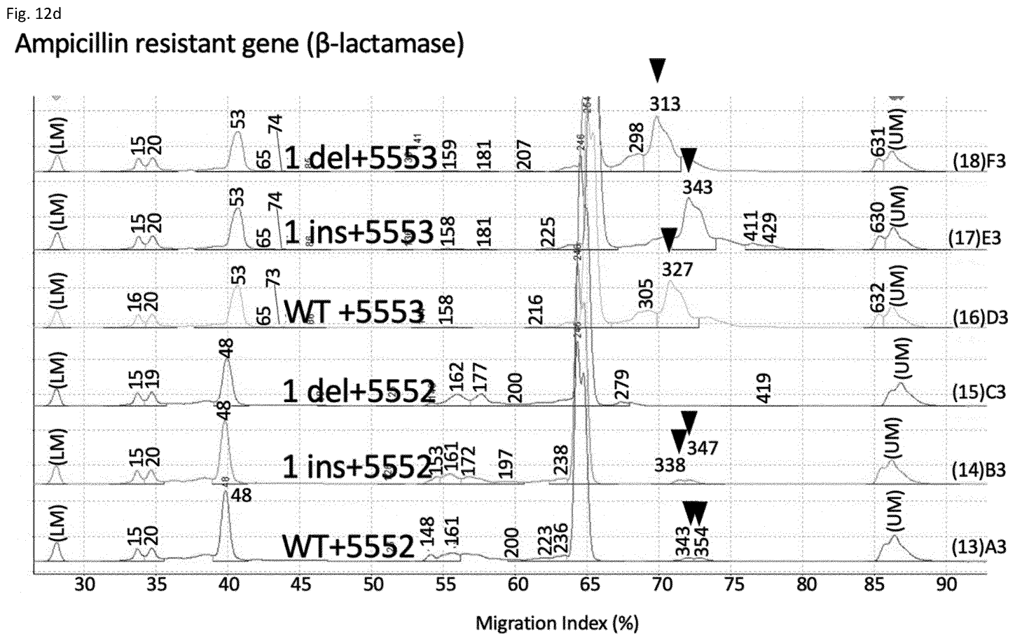

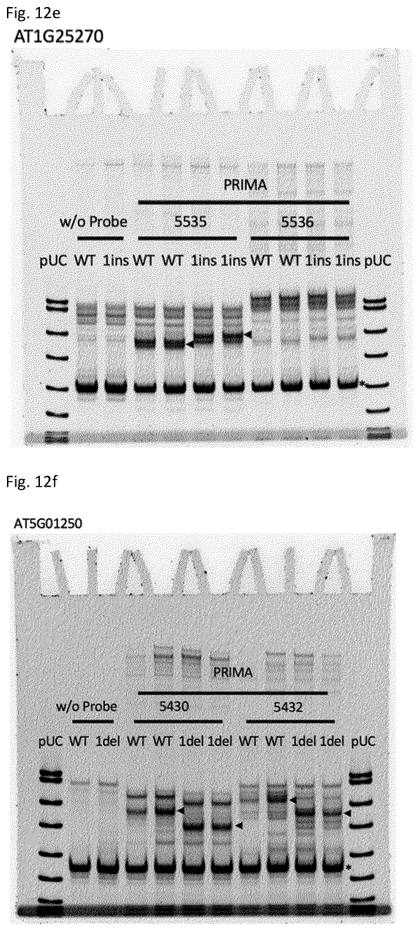

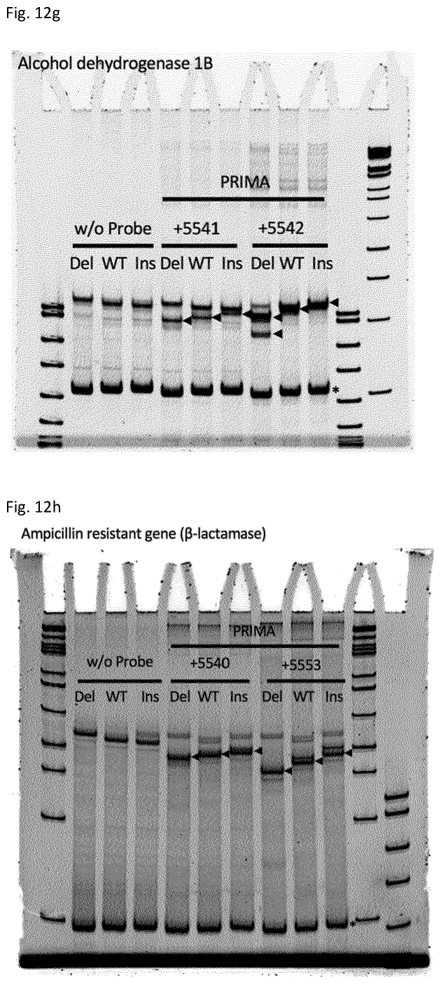

On the other hand, prePRIMA and PRIMA is able to distinguish the genotypes with a single run (FIG. 11 and FIG. 10). When using 5 bp deletion sequence as a probe, heteroduplex peaks derived from wild type homozygous or mutant homozygous were observed with different mobility shifts. The heterozygous sample showed both peaks (FIG. 10c and FIG. 10d). Taken together, prePRIMA and PRIMA save the costs, labor work and/or time for genotyping compared with HMA. PRIMA does not require synthesizing a long probe compared to prePRIMA and is therefore recommend as the best method for genotyping.

### Example 6: PRIMA is Applicable to Many Sequences

The inventors tested whether PRIMA is available for several sequences from plants, bacteria and human. They successfully detected heteroduplex peaks with different sizes from each genotype and materials with PRIMA (and prePRIMA). (FIG. 13).

When the inventors encountered a case that a peak pattern with a short single-stranded DNA (sssDNA) probe (forward probe) was not very clearly distinguishable, they tried another strand of sssDNA (reverse probe). The same PCR fragment and the same probe region was tested with a complementary sequence as a probe. Different mobility of heteroduplex peak was detected by using a forward or reverse probe (FIG. 13). This result is compatible with the case of HMA in Bhattacharyya and Lilley, 1989 NAR. Different peaks were detected by complementary probe. Normally, at least one of these two probes showed a clear difference with different genotype (FIG. 13). If both strands did not work, a slight shift of the probe position was performed.

### Example 7: PRIMA is Possible to Distinguish Type of Base (A, T, G and C)

Recent development of CRISPR system enabled to ‘base-editing’ using nuclease-inactive version of SpCas9 (Kumor et al., Nature 2016, Nishida et al., Science 2016, Nishimasu et al., 2018). To test whether PRIMA is usable to distinguish type of base, the inventors performed PRIMA (FIG. 13). They could distinguish A or T at the same position (FIG. 13b). This result even broadens the possibility of application of PRIMA for single nucleotide polymorphism (SNP) typing besides indel detection. SNP typing can be also useful for the chemically mutagenized genotype (such as EMS-mutagenized lines in plant). Homeologs might be distinguished by PRIMA.

### Methods

### Protocol for PRIMA Using MultiNA DNA-500 Kit (FIG. 15)

1. Set up a PCR condition based on the target site of genome editing.
   Design primers which satisfy the criteria below.
   Forward primer position: about 100 bp upstream of the (putative) mutation position.
   Reverse primer position: about 100 bp downstream of the (putative) mutation position.
   It is recommended to design these primers with the product size ranged between 180-220 bp.
2. Design a probe containing 5 bp deletion around the (putative) mutation position PRIMA is working with short single-stranded DNA (sssDNA). We confirmed 40mer sssDNA is long enough to introduce the conformational change after the re-annealing process in step4. We recommended probe position 5 bp deletion starting from −6 to −2 from of PAM sequence; see FIG. 15)
3. PCR
   Prepare PCR fragment with normal PCR protocol using the primers in step1.
4. Preparation of the mixture of PCR product and probe and re-annealing
   Mix the 9 μl of PCR product and 1 μl of 10 μM probe you prepared in step2.
   Then, preform denaturation and re-annealing reaction as follows; 5 min. at 95° C., cooling to 25° C. at 0.1° C. per second.
5. Detect heteroduplex peak
   Heteroduplex peak(s) can be detected by MultiNA, Microchip Electrophoresis System from SHIMADZU. This detection step can be achieved by polyacrylamide gel electrophoresis (Ota et al., 2013 Genes Cells, Ansai et al., 2014 Dev Growth Differ, Delwart et al., 1993 Science) or other high resolution electrophoresis machine (i.e. QIAxcel by Qiagen).

1. A method for distinguishing a first nucleic acid sequence from a second nucleic acid sequence by electrophoresis,
wherein
the first nucleic acid sequence S1 comprises
a first 5′ common sequence tract C1, and
a first variable sequence tract V1 of 1 to 10 nucleotides, immediately adjacent in 3′ direction to C1; and
a first 3′ common sequence tract C2 positioned in 3′ direction of C1;
the second nucleic acid sequence S2 comprises
a second 5′ common sequence tract C1′, and
a second, optional, variable sequence tract V2 of 1 to 10 nucleotides, immediately adjacent in 3′ direction to C1′; and
a second 3′ common sequence tract C2′ positioned in 3′ direction of C1′;
and wherein
the first 5′ common sequence tract C1 is identical to the second 5′ common sequence tract C1′, or
C1′ is 1 to 9 nucleotides shorter at the 3′ end than C1 and C1′ is identical to C1 from the 5′ end of C1/C1′; and
the first 3′ common sequence tract C2 is identical to the second 3′ common sequence tract C2′, or
C2′ is 1 to 9 nucleotides shorter at the 5′ end than the first 3′ common sequence tract C2 and C2′ is identical to C2 from the 3′ end of C2/C2′; and
with the proviso that S1 and S2 with respect to their sequence tracts C1-V1-C2 and C1′-V2-C2′ differ from each other in length by ≤10 nucleotides;
said method comprising:
contacting the first nucleic acid sequence and the second nucleic acid sequence with a probe sequence P, said probe sequence consisting, in 5′ to 3′ orientation, of a sequence RC2 that is reverse complementary to the 3′ common sequence tract C2 and a sequence RC1 that is reverse complementary to the 5′ common sequence tract C1,
under conditions allowing the hybridization of the probe sequence to the first and second nucleic acid sequence, thereby forming a first probe hybrid and a second probe hybrid,
and subsequently submitting the first and second probe hybrids to electrophoresis and detecting the electrophoretic mobility of the first and second probe hybrid.

2. The method according to claim 1, wherein the length of the first nucleic acid sequence S1 and the length of the second nucleic acid sequence S2 is between 40 nucleotides and 3500 nucleotides, particularly between 150 and 250 nucleotides, more particularly between 180 and 220 nucleotides.

3. The method according to claim 1, wherein the first nucleic acid sequence S1 comprises at least (≥) 5, particularly ≥35, more particularly ≥47 nucleotides immediately adjacent in 5′ direction to the first 5′ common sequence tract C1 and at least 5, particularly ≥35, more particularly ≥47 nucleotides immediately adjacent in 3′ direction to the first 3′ common sequence tract C2 and the second nucleic acid sequence S2 comprises at least 5, particularly ≥35, more particularly ≥47 nucleotides immediately adjacent in 5′ direction to second 5′ common sequence tract C1′ and at least 5, particularly ≥35, more particularly ≥47 nucleotides immediately adjacent in 3′ direction to the second 3′ common sequence tract C2′.

4. The method according to claim 1, wherein the total length of the sum of the first 5′ common sequence tract C1 and the first 3′ common sequence tract C2 is between 18 and 3500 nucleotides, particularly between 18 and 80 nucleotides.

5. The method according to claim 1, wherein the ratio between the length of the first 5′ common sequence tract C1 and the length of the first 3′ common sequence tract C2 is between 1:7 to 7:1, particularly between 3:5 and 5:3, more particularly 1:1, wherein the minimum length of the first 5′ common sequence tract C1 and of the first 3′ common sequence tract C2 is 5 nucleotides.

6. The method according to claim 1, wherein the first variable sequence tract V1 and the second variable sequence tract V2 have independently from each other a length between 4 and 10 nucleotides, particularly between 4 and 6 nucleotides.

7. The method according to claim 1, wherein the first variable sequence tract V1 differs from the second variable sequence tract V2 in length and/or the base sequence and/or composition of the first variable sequence tract V1 differs from the base sequence and/or composition of the second variable sequence tract V2 in at least one position.

8. The method according to claim 1, wherein the length of the first variable sequence V1 tract differs from the length of the second variable sequence tract V2 in ≤10 nucleotides, particularly in ≤2 nucleotides, more particularly in one nucleotide.

9. The method according to claim 1, wherein the composition of the first variable sequence tract V1 differs from the composition of the second variable sequence tract V2 in two positions, particularly in one position.

10. The method according to claim 1, wherein the first nucleic acid sequence S1 is hybridized to its reverse complementary sequence, and/or the second nucleic acid sequence S2 is hybridized to its reverse complementary sequence.

11. The method according to claim 1, wherein the probe sequence P is hybridized to its reverse complementary sequence.

12. The method according to claim 1, wherein the first probe hybrid and the second probe hybrid are obtained by applying a temperature above the melting point of the first and second nucleic acid sequence followed by applying a temperature below the melting point of the probe sequence.


SEQUENCE LISTING (73 sequences)

SEQ ID NO: 1 - DNA, 48 bp, Arabidopsis thaliana
ctgcagaagatgaactccgttctggtatctacaaagtctccaaggttt

SEQ ID NO: 2 - DNA, 21 bp, Arabidopsis thaliana
gaactccgttctggtatctac

SEQ ID NO: 3 - DNA, 20 bp, Artificial Sequence
  base deletion
gaactccttctggtatctac

SEQ ID NO: 4 - DNA, 19 bp, Arabidopsis thaliana
gaactcctctggtatctac

SEQ ID NO: 5 - DNA, 18 bp, Artificial Sequence
  base deletion
gaactccctggtatctac

SEQ ID NO: 6 - DNA, 17 bp, Artificial Sequence
  base deletion
gaactcctggtatctac

SEQ ID NO: 7 - DNA, 16 bp, Artificial Sequence
  base deletion
gaactccggtatctac

SEQ ID NO: 8 - DNA, 15 bp, Artificial Sequence
  base deletion
gaactccgtatctac

SEQ ID NO: 9 - DNA, 14 bp, Artificial Sequence
  base deletion
gaactcctatctac

SEQ ID NO: 10 - DNA, 49 bp, Arabidopsis thaliana
agcagctttcaacaacctccatggattcctcagagacccatgaagccat

SEQ ID NO: 11 - DNA, 22 bp, Arabidopsis thaliana
aacaacctccatggattcctca

SEQ ID NO: 12 - DNA, 21 bp, Artificial Sequence
  base deletion
aacaaccccatggattcctca

SEQ ID NO: 13 - DNA, 20 bp, Artificial Sequence
  base deletion
aacaacccatggattcctca

SEQ ID NO: 14 - DNA, 19 bp, Artificial Sequence
  base deletion
aacaaccatggattcctca

SEQ ID NO: 15 - DNA, 18 bp, Artificial Sequence
  base deletion
aacaacctggattcctca

SEQ ID NO: 16 - DNA, 17 bp, Artificial Sequence
  base deletion
aacaaccggattcctca

SEQ ID NO: 17 - DNA, 16 bp, Artificial Sequence
  base deletion
aacaaccgattcctca

SEQ ID NO: 18 - DNA, 15 bp, Artificial Sequence
  base deletion
aacaaccattcctca

SEQ ID NO: 19 - DNA, 18 bp, Arabidopsis thaliana
actccgttctggtatcta

SEQ ID NO: 20 - DNA, 17 bp, Artificial Sequence
  base deletion
actccttctggtatcta

SEQ ID NO: 21 - DNA, 16 bp, Artificial Sequence
  base deletion
actcctctggtatcta

SEQ ID NO: 22 - DNA, 15 bp, Artificial Sequence
  base deletion
actccctggtatcta

SEQ ID NO: 23 - DNA, 14 bp, Artificial Sequence
  base deletion
actcctggtatcta

SEQ ID NO: 24 - DNA, 13 bp, Artificial Sequence
  base deletion
actccggtatcta

SEQ ID NO: 25 - DNA, 12 bp, Artificial Sequence
  base deletion
actccgtatcta

SEQ ID NO: 26 - DNA, 11 bp, Artificial Sequence
  base deletion
actcctatcta

SEQ ID NO: 27 - DNA, 17 bp, Arabidopsis thaliana
caacctccatggattcc

SEQ ID NO: 28 - DNA, 16 bp, Artificial Sequence
  base deletion
caaccccatggattcc

SEQ ID NO: 29 - DNA, 15 bp, Artificial Sequence
  base deletion
caacccatggattcc

SEQ ID NO: 30 - DNA, 14 bp, Artificial Sequence
  base deletion
caaccatggattcc

SEQ ID NO: 31 - DNA, 13 bp, Artificial Sequence
  base deletion
caacctggattcc

SEQ ID NO: 32 - DNA, 12 bp, Artificial Sequence
  base deletion
caaccggattcc

SEQ ID NO: 33 - DNA, 11 bp, Artificial Sequence
  base deletion
caaccgattcc

SEQ ID NO: 34 - DNA, 10 bp, Artificial Sequence
  base deletion
caaccattcc

SEQ ID NO: 35 - DNA, 16 bp, Artificial Sequence
  base deletion
tttcaacaacccatgg

SEQ ID NO: 36 - DNA, 17 bp, Artificial Sequence
  base deletion
tttcaacaaccccatgg

SEQ ID NO: 37 - DNA, 18 bp, Arabidopsis thaliana
tttcaacaacctccatgg

SEQ ID NO: 38 - DNA, 18 bp, Artificial Sequence
  base insertion
tttcaacaacctccatgg

SEQ ID NO: 39 - DNA, 23 bp, Artificial Sequence
  DNA fragment with deletion
agaagatgaactccctacaaagt

SEQ ID NO: 40 - DNA, 24 bp, Artificial Sequence
  DNA fragment with deletion
agaagatgaactcctctacaaagt

SEQ ID NO: 41 - DNA, 25 bp, Artificial Sequence
  DNA fragment with deletion
agaagatgaactccatctacaaagt

SEQ ID NO: 42 - DNA, 26 bp, Artificial Sequence
  DNA fragment with deletion
agaagatgaactcctatctacaaagt

SEQ ID NO: 43 - DNA, 27 bp, Artificial Sequence
  DNA fragment with deletion
agaagatgaactccgtatctacaaagt

SEQ ID NO: 44 - DNA, 28 bp, Artificial Sequence
  DNA fragment with deletion
agaagatgaactccggtatctacaaagt

SEQ ID NO: 45 - DNA, 29 bp, Artificial Sequence
  DNA fragment with deletion
agaagatgaactcctggtatctacaaagt

SEQ ID NO: 46 - DNA, 30 bp, Artificial Sequence
  DNA fragment with deletion
agaagatgaactccctggtatctacaaagt

SEQ ID NO: 47 - DNA, 31 bp, Artificial Sequence
  DNA fragment with deletion
agaagatgaactcctctggtatctacaaagt

SEQ ID NO: 48 - DNA, 32 bp, Artificial Sequence
  DNA fragment with deletion
agaagatgaactccttctggtatctacaaagt

SEQ ID NO: 49 - DNA, 33 bp, Arabidopsis thaliana
agaagatgaactccgttctggtatctacaaagt

SEQ ID NO: 50 - DNA, 34 bp, Artificial Sequence
  DNA fragment with insertion
agaagatgaactccgattctggtatctacaaagt

SEQ ID NO: 51 - DNA, 35 bp, Artificial Sequence
  DNA fragment with insertion
agaagatgaactccgaattctggtatctacaaagt

SEQ ID NO: 52 - DNA, 36 bp, Artificial Sequence
  DNA fragment with insertion
agaagatgaactccgaaattctggtatctacaaagt

SEQ ID NO: 53 - DNA, 37 bp, Artificial Sequence
  DNA fragment with insertion
agaagatgaactccgaaaattctggtatctacaaagt

SEQ ID NO: 54 - DNA, 38 bp, Artificial Sequence
  DNA fragment with insertion
agaagatgaactccgaaaaattctggtatctacaaagt

SEQ ID NO: 55 - DNA, 39 bp, Artificial Sequence
  DNA fragment with insertion
agaagatgaactccgaaaaaattctggtatctacaaagt

SEQ ID NO: 56 - DNA, 40 bp, Artificial Sequence
  DNA fragment with insertion
agaagatgaactccgaaaaaaattctggtatctacaaagt

SEQ ID NO: 57 - DNA, 41 bp, Artificial Sequence
  DNA fragment with insertion
agaagatgaactccgaaaaaaaattctggtatctacaaagt

SEQ ID NO: 58 - DNA, 42 bp, Artificial Sequence
  DNA fragment with insertion
agaagatgaactccgaaaaaaaaattctggtatctacaaagt

SEQ ID NO: 59 - DNA, 43 bp, Artificial Sequence
  DNA fragment with insertion
agaagatgaactccgaaaaaaaaaattctggtatctacaaagt

SEQ ID NO: 60 - DNA, 61 bp, Artificial Sequence
  base-edited sequence
ctcttggtcgttctgcagaagatgaactccgattctggtatctacaaagtctccaaggttt

SEQ ID NO: 61 - DNA, 61 bp, Artificial Sequence
  Gene construction of RDP1
ggtcgttctgcagaagatgaactccgattctggtatctacaaagtctccaaggtttgtgta

SEQ ID NO: 62 - DNA, 60 bp, Arabidopsis thaliana
ggtcgttctgcagaagatgaactccgttctggtatctacaaagtctccaaggtttgtgta

SEQ ID NO: 63 - DNA, 59 bp, Artificial Sequence
  Gene construction of RDP1
ggtcgttctgcagaagatgaactccttctggtatctacaaagtctccaaggtttgtgta

SEQ ID NO: 64 - DNA, 23 bp, Arabidopsis thaliana
gcagaagatgaactccgttctgg

SEQ ID NO: 65 - DNA, 40 bp, Artificial Sequence
  sssProbe
gttctgcagaagatgaactctggtatctacaaagtctcaa

SEQ ID NO: 66 - DNA, 305 bp, Arabidopsis thaliana
taggcacaatggaaagttagtttctttgtccttcttctggttgatgttagaattacttgaatgttatgactgactcggttcttatttgtctaggttcttcctaggttcgaacaaagtgatgcaggttgctcttggtcgttctgcagaagatgaactccgttctggtatctacaaagtctccaaggtttgtgtattctgcttcttacaatggttcttttatgttaaatggtcattttttgtcagttagatttacatatgttgtggaatgttgtttcagctgcttcgtggtgatactggacttcttg

SEQ ID NO: 67 - DNA, 296 bp, Arabidopsis thaliana
atagaaagttccaagctttttctcaaatggttctgatttaagtaagagtgaagaaaagtaaaaatagagtcagaaatggagaaacagaggagagaagaaagcagctttcaacaacctccatggattcctcagacacccatgaagccattttcaccgatctgcccatacacggtggaggatcaatatcatagcagtcaattggaggaaaggtttgtgcttttttgttctaaagttgagaaatttcaaagagtagtgatgggtaattggttaagtaaggtattgatgcatgcaggaga

SEQ ID NO: 68 - DNA, 200 bp, Arabidopsis thaliana
tatttgtctaggttcttcctaggttcgaacaaagtgatgcaggttgctcttggtcgttctgcagaagatgaactccgttctggtatctacaaagtctccaaggtttgtgtattctgcttcttacaatggttcttttatgttaaatggtcattttttgtcagttagatttacatatgttgtggaatgttgtttcagctgct

SEQ ID NO: 69 - DNA, 200 bp, Arabidopsis thaliana
tcaaatggttctgatttaagtaagagtgaagaaaagtaaaaatagagtcagaaatggagaaacagaggagagaagaaagcagctttcaacaacctccatggattcctcagacacccatgaagccattttcaccgatctgcccatacacggtggaggatcaatatcatagcagtcaattggaggaaaggtttgtgcttttt

SEQ ID NO: 70 - DNA, 274 bp, Arabidopsis thaliana
tcaaatggttctgatttaagtaagagtgaagaaaagtaaaaatagagtcagaaatggagaaacagaggagagaagaaagcagctttcaacaacctccatggattcctcagacacccatgaagccattttcaccgatctgcccatacacggtggaggatcaatatcatagcagtcaattggaggaaaggtttgtgcttttttgttctaaagttgagaaatttcaaagagtagtgatgggtaattggttaagtaaggtattgatgcatgcaggaga

SEQ ID NO: 71 - DNA, 225 bp, Arabidopsis thaliana
taggcacaatggaaagttagtttctttgtccttcttctggttgatgttagaattacttgaatgttatgactgactcggttcttatttgtctaggttcttcctaggttcgaacaaagtgatgcaggttgctcttggtcgttctgcagaagatgaactccgttctggtatctacaaagtctccaaggtttgtgtattctgcttcttacaatggttcttttatgttaa

SEQ ID NO: 72 - DNA, 225 bp, Arabidopsis thaliana; identical to SEQ ID NO: 71

SEQ ID NO: 73 - DNA, 225 bp, Arabidopsis thaliana; identical to SEQ ID NO: 71